(19) United States
(12) Patent Application Publication
(10) Pub. No.: US 2024/0347198 A1
(43) Pub. Date: Oct. 17, 2024

(54) REAL-TIME CORRECTIVE ACTIONS FOR OXYGEN SATURATION PREDICTIONS

(71) Applicant: Covidien LP, Mansfield, MA (US)

(72) Inventors: Dean Montgomery, Edinburgh (GB); Paul S. Addison, Edinburgh (GB)

(21) Appl. No.: 18/626,077

(22) Filed: Apr. 3, 2024

Related U.S. Application Data
(60) Provisional application No. 63/495,731, filed on Apr. 12, 2023.

(51) Int. Cl. G16H 50/20; A61B 5/145

(52) U.S. Cl. CPC G16H 50/20; A61B 5/14542

(57) ABSTRACT

In some examples, a computing system tracks, across a plurality of predictions, prediction performance of a first oxygen saturation prediction model used by one or more patient monitoring devices by comparing a respective prediction made by the first oxygen saturation prediction model to a corresponding ground truth. The computing system determines whether the prediction performance of the first oxygen saturation prediction model meets a performance metric, wherein the performance metric includes an accuracy level, a specificity level, a sensitivity level, or any combination thereof. The computing system may, in response to determining that the prediction performance of the first oxygen saturation prediction model does not meet the performance metric, cause the one or more patient monitoring devices to switch to a second oxygen saturation prediction model to predict future oxygen saturation levels of one or more patients.

### CROSS-REFERENCE TO RELATED APPLICATION

This application claims priority to and the benefit of U.S. Provisional Patent Application No. 63/495,731, entitled “REAL-TIME CORRECTIVE ACTIONS FOR OXYGEN SATURATION PREDICTIONS,” filed on Apr. 12, 2023, which is incorporated herein by reference in its entirety for all purposes.

### TECHNICAL FIELD

This disclosure relates to patient monitoring.

### BACKGROUND

Oxygen saturation monitoring systems are configured to monitor the oxygen saturation levels of a patient. For example, a pulse oximetry sensor placed on a patient can produce a photoplethysmogram that indicates the blood oxygen saturation level of the patient. When the oxygen saturation level of the patient decreases to reach a desaturation threshold, the oxygen saturation monitoring system may output an indication that the patient is experiencing oxygen desaturation.

### SUMMARY

The present disclosure describes example devices, systems, and techniques for patient monitoring and increasing the accuracy of oxygen saturation prediction models used by patient monitoring devices, such as oxygen saturation monitoring devices, to predict the future oxygen saturation levels of patients. In particular, patient monitoring device that monitors the oxygen saturation levels of a patient may, in response to the oxygen saturation level of the patient decreasing to reach a desaturation threshold, use an oxygen saturation prediction model to predict whether the oxygen saturation level of the patient will return above the desaturation threshold by the end of a predefined period. If the oxygen saturation prediction model predicts that the oxygen saturation level of the patient will return back above the desaturation threshold by the end of the predefined period, then the patient monitoring device may refrain from outputting an indication that the patient is experiencing oxygen desaturation. It should be appreciated that, as used herein, “to predict the future oxygen saturation levels of the patient” means or includes “to predict whether, after the oxygen saturation levels of the patient decrease to reach a desaturation threshold, the oxygen saturation levels of the patient will return above the desaturation threshold by the end of a predefined period.”

A computing system may track the prediction performance of an oxygen saturation prediction model used by one or more patient monitoring devices to predict the future oxygen saturation levels of patients (e.g., to predict whether, after the oxygen saturation levels of each patient decreases to reach a desaturation threshold, the oxygen saturation levels of each patient will return above the desaturation threshold by the end of a predefined period). If the computing system determines that the prediction performance of an oxygen saturation prediction model does not meet a performance metric, then the computing system may perform one or more corrective actions that may enable the one or more patient monitoring devices to more accurately predict the future oxygen saturation levels of patients (e.g., to more accurately predict whether, after the oxygen saturation levels of each patient decreases to reach a desaturation threshold, the oxygen saturation levels of each patient will return above the desaturation threshold by the end of a predefined period).

For example, the computing system may update the oxygen saturation prediction model to improve the accuracy of predictions made by the model, such as by training the oxygen saturation prediction model using data (e.g., ground truth data) associated with incorrect predictions made by the model as training data. In another example, the computing system may switch the oxygen saturation prediction model with which the one or more patient monitoring devices monitor and predict oxygen saturation levels of patients. By performing one or more corrective actions in response to determining that the prediction performance of an oxygen saturation prediction model does not meet a performance metric, the techniques described herein may improve the functioning of a patient monitoring device that uses the oxygen saturation prediction model by potentially increasing the accuracy of the predictions of the future oxygen saturation levels of patients made by the patient monitoring device.

In some aspects, the techniques described herein relate to a method including: tracking, with processing circuitry of a computing system and across a plurality of predictions, prediction performance of a first oxygen saturation prediction model used by one or more patient monitoring devices to predict future oxygen saturation levels of one or more patients; determining, with the processing circuitry, that the prediction performance of the first oxygen saturation prediction model does not meet a performance metric; and in response to determining that the prediction performance of the first oxygen saturation prediction model does not meet the performance metric, cause, with the processing circuitry, the one or more patient monitoring devices to use a second oxygen saturation prediction model to predict the future oxygen saturation levels of the one or more patients.

In some aspects, the techniques described herein relate to a computing system including: memory; and processing circuitry configured to: track, across a plurality of predictions, prediction performance of a first oxygen saturation prediction model used by one or more patient monitoring devices to predict future oxygen saturation levels of one or more patients; determine that the prediction performance of the first oxygen saturation prediction model does not meet a performance metric; and in response to determining that the prediction performance of the first oxygen saturation prediction model does not meet the performance metric, cause the one or more patient monitoring devices to use a second oxygen saturation prediction model to predict the future oxygen saturation levels of the one or more patients.

In some aspects, the techniques described herein relate to an apparatus including: means for tracking, across a plurality of predictions, prediction performance of a first oxygen saturation prediction model used by one or more patient monitoring devices to predict future oxygen saturation levels of one or more patients; means for determining that the prediction performance of the first oxygen saturation prediction model does not meet a performance metric; and means for, in response to determining that the prediction performance of the first oxygen saturation prediction model does not meet the performance metric, causing the one or more patient monitoring devices to use a second oxygen saturation prediction model to predict the future oxygen saturation levels of the one or more patients.

In some aspects, the techniques described herein relate to a non-transitory computer readable storage medium comprising instructions that, when executed by one or more processors of a computing system, cause the one or more processors to: track, across a plurality of predictions, prediction performance of a first oxygen saturation prediction model used by one or more patient monitoring devices to predict future oxygen saturation levels of one or more patients; determine that the prediction performance of the first oxygen saturation prediction model does not meet a performance metric; and in response to determining that the prediction performance of the first oxygen saturation prediction model does not meet the performance metric, cause the one or more patient monitoring devices to use a second oxygen saturation prediction model to predict the future oxygen saturation levels of the one or more patients.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 illustrates an example environment in which an example patient monitoring device monitors the oxygen saturation levels of a patient, in accordance with an aspect of the present disclosure;

FIG. 2 is a conceptual block diagram illustrating an example environment in which an example computing system may taking corrective action in response to the prediction performance of an example oxygen saturation prediction model failing to meet a performance metric, in accordance with an aspect of the present disclosure;

FIG. 3 is a block diagram illustrating an example computing system, in accordance with one or more aspects of the present disclosure, in accordance with an aspect of the present disclosure;

FIG. 4 illustrates an example deep learning architecture of one or more of oxygen saturation prediction models of FIG. 2, in accordance with an aspect of the present disclosure;

FIG. 5 is a flow diagram illustrating an example method for taking one or more corrective actions based on the predictive performance of an oxygen saturation prediction model, in accordance with an aspect of the present disclosure;

FIG. 6 is a flow diagram illustrating an example method for switching oxygen saturation models to increase the accuracy of predictions made by patient monitoring devices, in accordance with an aspect of the present disclosure;

FIG. 7 is a flow diagram illustrating an example method for generating an updated oxygen saturation prediction model to increase accuracy of predictions made by patient monitoring devices, in accordance with an aspect of the present disclosure; and

FIG. 8 is a flow diagram illustrating an example method for retraining an oxygen saturation prediction model using ground truth values, in accordance with an aspect of the present disclosure.

### DETAILED DESCRIPTION

Some patient monitoring devices are configured to monitor the oxygen saturation level of a patient via an oxygen sensor (e.g., a pulse oximeter or a region oxygen saturation sensor). When the sensed oxygen saturation level of a patient decreases to reach a desaturation threshold, the patient monitoring device may, instead of outputting an indication that the patient is experiencing oxygen desaturation, use an oxygen saturation prediction model to predict whether the oxygen saturation level of the patient will increase back above the desaturation threshold within a predefined time period. In general, the present disclosure describe devices, systems, and techniques for taking corrective action in response to an oxygen saturation prediction model failing to meet a performance metric. The performance metric can indicate, for example, that a device implementing the oxygen saturation prediction model is not adequately (e.g., accurately) predicting the future oxygen saturation levels of a patient (e.g., not adequately predicting whether, after the oxygen saturation levels of the patient decreases to reach the desaturation threshold, the oxygen saturation levels of the patient will return above the desaturation threshold by the end of a predefined time period).

If the patient monitoring device, using the oxygen saturation prediction model, predicts that the oxygen saturation level of the patient will not increase back above the desaturation threshold within the predefined time period, then the patient monitoring device may output an indication that the patient is experiencing oxygen desaturation. If the patient monitoring device predicts that the oxygen saturation level of the patient will increase back above the desaturation threshold within the predefined time period, then the patient monitoring device may refrain from outputting an indication that the patient is experiencing oxygen desaturation. Instead, at the end of the predefined time period, the patient monitoring device may determine whether the oxygen saturation level of the patient has indeed increased back above the desaturation threshold. If the oxygen saturation level of the patient has not increased back above the desaturation threshold at the end of the predefined time period, then the patient monitoring device may, at the end of the predefined time period, output an indication that the patient is experiencing oxygen desaturation.

By predicting whether the oxygen saturation level of the patient will increase back above the desaturation threshold within a predefined time period, the patient monitoring device predicts, when the oxygen saturation level of a patient decreases to reach a desaturation threshold, whether the patient is experiencing a trivial desaturation event in which the oxygen saturation level of the patient may only briefly dip below the desaturation threshold before increasing back above the desaturation threshold, or whether the patient is experiencing a non-trivial desaturation event that may require clinical intervention. Because such trivial desaturation events may not be medically meaningful, e.g., may not be indicative of the occurrence of a medical event requiring intervention, outputting indications that the patient is experiencing oxygen desaturation in response to the patient experiencing such trivial desaturation events may not provide useful information to a clinician monitoring the health of the patient, and the indications may be a nuisance that unnecessarily distracts the clinician from performing other tasks. As such, by using an oxygen saturation prediction model to predict whether the oxygen saturation level of the patient will increase back above the desaturation threshold within a predefined time period in response to the oxygen saturation level of the patient decreases to reach the desaturation threshold instead of immediately outputting an alert may reduce the number of times the patient monitoring device outputs nuisance alerts indicative of trivial oxygen desaturation events, while still providing timely indications of more non-trivial desaturation events.

However, an oxygen saturation prediction model may not always accurately predict whether the oxygen saturation level of a patient will increase back above the desaturation threshold within a predefined time period. For example, an oxygen saturation prediction model may, when the oxygen saturation level of a patient decreases to reach a desaturation threshold, incorrectly predict that the oxygen saturation level of the patient will not increase back above the desaturation threshold within a predefined time period, which may cause a patient monitoring device to incorrectly output an alert indicative of a non-trivial desaturation event.

In accordance with aspects of this disclosure, a computing system is configured to track, across a plurality of predictions made by an oxygen saturation prediction model, the prediction performance of the oxygen saturation prediction model to determine whether the prediction performance of the oxygen saturation prediction model meets a performance metric. For example, the computing system may determine the accuracy level of the predictions made by the oxygen saturation prediction model and may determine whether the accuracy level meets a minimum accuracy level.

If the computing system determines that the prediction performance of the oxygen saturation prediction model does not meet the performance metric, then the computing system may perform one or more corrective actions to increase the accuracy of predictions made by patient monitoring devices the use the oxygen saturation prediction model. For example, the computing system may cause the patient monitoring devices that were using the oxygen saturation prediction model to switch to a different oxygen saturation prediction model to predict the future oxygen saturation levels of patients. In another example, the computing system may update the oxygen saturation prediction model to increase the prediction performance of the oxygen saturation prediction model, e.g., for a specific patient or population (e.g., a particular demographic of patients, a particular part of a day, a particular location of patients, or the like), such as by using ground truth data collected by the patient monitoring device(s) during patient monitoring session(s). In this way, the computing system may enable patient monitoring devices to more accurately predict the future oxygen saturation levels of patients.

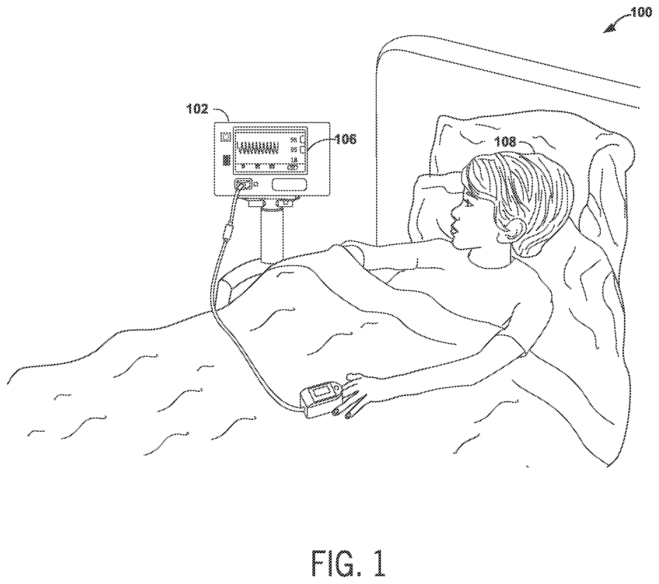

FIG. 1 illustrates an example environment 100 in which an example patient monitoring device 102 monitors the oxygen saturation levels of a patient 108. As shown in FIG. 1, patient monitoring device 102 may be any suitable patient monitoring device configured to determine (e.g., sense) an oxygen saturation (e.g., blood oxygen saturation or regional oxygen saturation) of patient 108. For example, patient monitoring device 102 may include one or more oxygen saturation sensing devices configured to generate a plethysmography (PPG) based on a signal from an optical sensor coupled to patient 108, where the PPG indicates the blood oxygen saturation, such as SpO₂, of patient 108.

In some examples, patient monitoring device 102 is configured to continuously monitor and output (e.g., display) the oxygen saturation level of patient 108, e.g., during a medical procedure or for more long-term monitoring, such as intensive care unit (ICU) and general post-operation monitoring. For example, patient monitoring device 102 may display the real-time oxygen saturation level of patient 108 at display device 106. A clinician may receive, from patient monitoring device 102 (e.g., by viewing display device 106), information regarding the oxygen saturation level of patient 108, and may adjust treatment or therapy to patient 108 based on the information. Patient monitoring device 102 may, for example, output the oxygen saturation level of patient 108 for display at display device 106 in graphical form, such as a graph of the oxygen saturation level of patient 108 over time, in textual form, such as outputting the oxygen saturation values of patient 108 for display at display device 106, in audible form via a speaker, such as sounds indicative of the oxygen saturation level of patient 108, and the like.

Patient monitoring device 102 is configured to use an oxygen saturation prediction model to predict the future oxygen saturation levels of patient 108. Specifically, when patient monitoring device 102 determines that the oxygen saturation level of patient 108 decreases to reach a desaturation threshold, patient monitoring device 102 may be configured to use an oxygen saturation prediction model to determine whether to output a notification indicative of patient 108 experiencing an oxygen desaturation event. Such a notification may be in graphical form, audible form, and the like.

Patient monitoring device 102 may be configured to, in response to the oxygen saturation level of patient 108 decreasing to reach a desaturation threshold, use an oxygen saturation prediction model to predict whether the oxygen saturation level of the patient will increase above the desaturation threshold by the end of a predetermined time period. Patient monitoring device 102 may be configured to, in response to predicting that the oxygen saturation level of the patient will increase above the desaturation threshold by the end of the predetermined time period, refrain from outputting a notification indicative of patient 108 experiencing an oxygen desaturation event.

In accordance with aspects of this disclosure, the prediction performance of an oxygen saturation prediction model used by patient monitoring device 102 may be tracked to determine whether the prediction performance of the oxygen saturation prediction model used by patient monitoring device 102 meets a performance metric. If the prediction performance of the oxygen saturation prediction model does not meet the performance metric, patient monitoring device 102 may be configured to stop using the oxygen saturation prediction model and to use a different oxygen saturation prediction model to predict the future oxygen saturation levels of patient 108, or to take other correction action, as described in more detail below.

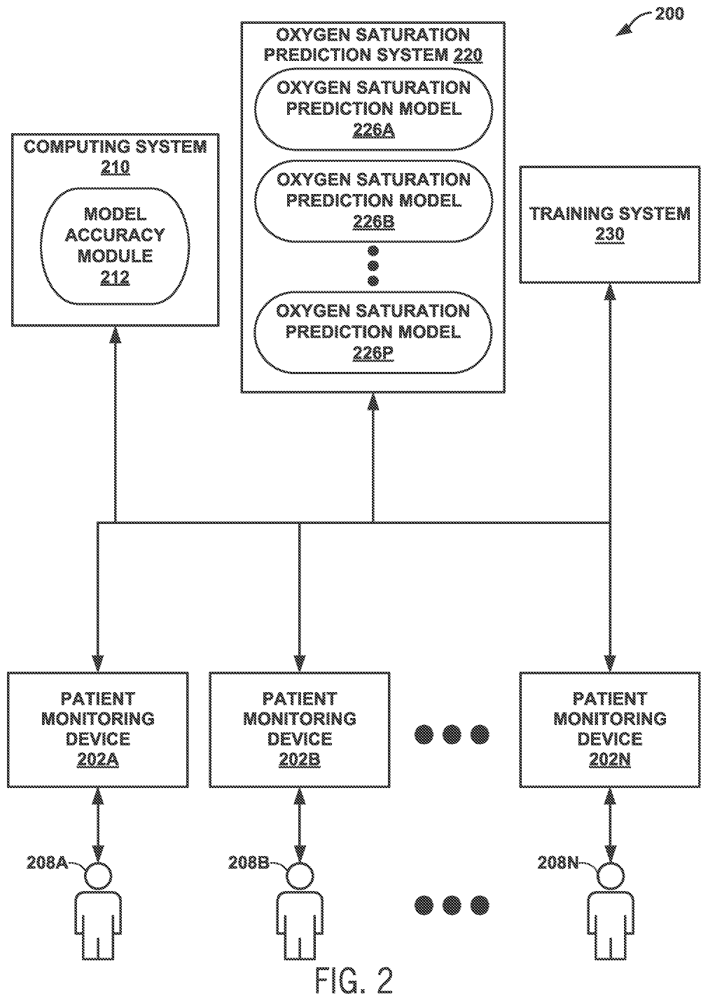

FIG. 2 is a conceptual block diagram illustrating an example environment 200 in which an example computing system may taking corrective action in response to the prediction performance of an example oxygen saturation prediction model failing to meet a performance metric. In the example of FIG. 2, environment 200 includes computing system 210, oxygen saturation prediction system 220, training system 230, and patient monitoring devices 202A-102N (“patient monitoring devices 202”) that are configured to communicate with includes computing via any wired or wireless communication channels.

Each of patient monitoring devices 202A-102N (“patient monitoring devices 202”) is an example of patient monitoring device 102 of FIG. 1 and may be any suitable patient monitoring device configured to determine (e.g., sense) an oxygen saturation (e.g., blood oxygen saturation or regional oxygen saturation) of a patient. For example, each of patient monitoring devices 202 may include one or more oxygen saturation sensing devices configured to generate a plethysmography (PPG) based on a signal from an optical sensor coupled to a patient, where the PPG indicates the blood oxygen saturation, such as SpO₂, of the patient.

In some examples, each of patient monitoring devices 202 is configured to continuously monitor and output (e.g., display) the oxygen saturation level of a corresponding patient, e.g., during a medical procedure or for more long-term monitoring, such as intensive care unit (ICU) and general post-operation monitoring. A clinician may receive, from a patient monitoring device, information regarding the oxygen saturation level of a patient, and may adjust treatment or therapy to the patient based on the information. Each of patient monitoring devices 202 may, for example, output the oxygen saturation level of a corresponding patient in graphical form, such as a graph of the oxygen saturation level of the corresponding patient over time, in textual form, such as outputting the oxygen saturation values of the corresponding patient, in audible form, such as sounds indicative of the oxygen saturation level of the corresponding patient, and the like.

Computing system 210 represents any suitable computing system, such as one or more desktop computers, laptop computers, mainframes, servers, cloud computing systems, or the like, capable of sending and receiving information both to and from oxygen saturation prediction system 220, training system 230, and/or one or more patient monitoring devices 202. In some examples, computing system 210 represents cloud computing systems that provide access to their respective services via a cloud.

Computing system 210 includes model accuracy module 212. Model accuracy module 212 may perform operations described herein using software, hardware, firmware, or a mixture of hardware, software, and firmware residing in and/or executing at computing system 210. Computing system 210 may execute model accuracy module 212 with processing circuitry of one or more processors or devices, as virtual machines executing on underlying hardware, as one or more services of an operating system or computing platform, and/or as one or more executable programs at an application layer of a computing platform of computing system 210.

Training system 230 represents any suitable computing system, such as one or more desktop computers, laptop computers, mainframes, servers, cloud computing systems, or the like, capable of sending and receiving information both to and from computing system 210, oxygen saturation prediction system 220, and/or patient monitoring devices 202. In some examples, oxygen saturation prediction system 220 represents cloud computing systems that provide access to their respective services via a cloud.

Oxygen saturation prediction system 220 represents any suitable computing system, such as one or more desktop computers, laptop computers, mainframes, servers, cloud computing systems, or the like, capable of sending and receiving information both to and from computing system 210, training system 230, and/or patient monitoring devices 202. In some examples, oxygen saturation prediction system 220 represents cloud computing systems that provide access to their respective services via a cloud. Although FIG. 2 illustrates computing system 210, oxygen saturation prediction system 220, and training system 230 as separate systems, two or more of computing system 210, oxygen saturation prediction system 220, and training system 230 may, in some examples, be a part of the same computing system.

In the example shown in FIG. 2, oxygen saturation prediction system 220 includes a plurality of oxygen saturation prediction models 226A-126P (“oxygen saturation prediction models 226”). Oxygen saturation prediction models 226 may perform operations described herein using software, hardware, firmware, or a mixture of hardware, software, and firmware residing in and/or executing at oxygen saturation prediction system 220. Oxygen saturation prediction system 220 may execute oxygen saturation prediction models 226 with processing circuitry of multiple processors or multiple devices, as virtual machines executing on underlying hardware, as one or more services of an operating system or computing platform, and/or as one or more executable programs at an application layer of a computing platform of oxygen saturation prediction system 220.

Each of oxygen saturation prediction models 226 is configured to perform functions associated with predicting the future oxygen saturation levels of one or more patients (e.g., predicting whether, after the oxygen saturation levels of each patient decreases to reach a desaturation threshold, the oxygen saturation levels of each patient will return above the desaturation threshold by the end of a predefined time period). In certain embodiments, the oxygen saturation prediction models 226 may not calculate a numerical value of the oxygen saturation levels, but instead may merely predict (e.g., binary prediction; yes or no) whether the oxygen saturation levels will return above the desaturation threshold by the end of the predefined time period. Each of oxygen saturation prediction models 226 may be trained via machine training or via other artificial intelligence techniques, such as via training system 230, to predict the future oxygen saturation levels of a patient. For example, an oxygen saturation prediction model may include one or more neural network algorithms trained via machine learning to take one or more of: the oxygen saturation levels of a patient, the blood pressure of a patient, and/or one or more metrics derived from the PPG signals of a patient determined during a time period immediately preceding a prediction point (e.g., a desaturation time at which the oxygen saturation levels decrease to reach a desaturation threshold) as inputs to predict the oxygen saturation level of the patient at the end of the predefined time period.

A neural network algorithm, or artificial neural network, may include a trainable or adaptive algorithm utilizing nodes that define rules. For example, a respective node of a plurality of nodes may utilize a function, such as a non-linear function or if-then rules, to generate an output based on an input. A respective node of the plurality of nodes may be connected to one or more different nodes of the plurality of nodes along an edge, such that the output of the respective node includes the input of the different node. The functions may include parameters that may be determined or adjusted using a training set of inputs and desired outputs (e.g., ground truth data), such as, for example, a predetermined association between one or more signals, such as one or more of: the oxygen saturation levels of the patient, the blood pressure of the patient, and/or one or more metrics derived from the PPG signals of the patient, along with a learning rule, such as a backpropagation learning rule. The backpropagation learning rule may utilize one or more error measurements comparing the desired output to the output produced by the neural network algorithm to train the neural network algorithm by varying the parameters to minimize the one or more error measurements.

An example neural network includes a plurality of nodes, at least some of the nodes having node parameters. An input including one or more of: the oxygen saturation levels of the patient, the blood pressure of the patient, and/or one or more metrics derived from the PPG signals of the patient may be input to a first node of the neural network algorithm. In some examples, the input may include a plurality of inputs, each input into a respective node. The first node may include a function configured to determine an output based on the input and one or more adjustable node parameters. In some examples, the neural network may include a propagation function configured to determine an input to a subsequent node based on the output of a preceding node and a bias value. In some examples, a learning rule may be configured to modify one or more node parameters to produce a favored output. For example, the favored output may be constrained by one or more threshold values and/or to minimize one or more error measurements. The favored output may include an output of a single node, a set of nodes, or the plurality of nodes.

The neural network algorithm may iteratively modify the node parameters until the output includes the favored output. In this way, the oxygen saturation prediction model may iteratively evaluate outputs of the neural network algorithm and iteratively modify at least one of the node parameters based on the evaluation of the outputs of the neural network algorithm to predict the oxygen saturation level of a patient at or within a predefined future time period based on the modified neural network algorithm (e.g., to predict whether, after the oxygen saturation levels of the patient decreases to reach a desaturation threshold, the oxygen saturation levels of the patient will return above the desaturation threshold by the end of a predefined time period). In some examples, a neural network algorithm may enable the oxygen saturation prediction model to more accurately predict the oxygen saturation level of a patient compared to other techniques and/or reduce computational time and/or power required to predict future oxygen saturation levels of the patient. Different oxygen saturation prediction models 226 may differ in the types of training data used, the type of algorithms used, the type of training, the inputs, and the like such that different oxygen saturation prediction models 226 may perform differently in predicting the future oxygen saturation levels of patients 208.

In some examples, oxygen saturation prediction system 220 is configured to receive, from a patient monitoring device of patient monitoring devices 202, a request to predict the future oxygen saturation level of a patient, which may indicate one or more of: the oxygen saturation levels of a patient, the blood pressure of a patient, one or more metrics derived from the PPG signals of a patient, and/or other parameters (e.g., patient demographic information, such as gender and/or age; patient health conditions). Oxygen saturation prediction system 220 may be configured to, in response to receiving the request, execute one or more of oxygen saturation prediction models 226 to utilize the information indicated by the request as inputs to the one or more of oxygen saturation prediction models 226 to predict the future oxygen saturation level of the patient at the end of the predefined time period. For example, oxygen saturation prediction system 220 may be configured to execute one or more of oxygen saturation prediction models 226 to utilize the information indicated by the request as inputs to the one or more of oxygen saturation prediction models 226 to predict whether the oxygen saturation level of a patient will increase above the desaturation threshold by the end of a predetermined period.

In some examples, one or more of oxygen saturation prediction models 226 are trained to be specific to a particular manufacturer of patient monitoring devices and/or trained to be specific to a particular model of a patient monitoring device made by a particular manufacturer. That is, one or more of oxygen saturation prediction models 226 may be trained to be used by patient monitoring devices made by a particular manufacturer and/or be trained to be used by a particular model of patient monitoring devices made by a particular manufacturer.

An oxygen saturation prediction model can be trained to be specific to a particular manufacturer of patient monitoring devices using training data such as PPG signals and oxygen saturation levels received and/or determined by patient monitoring devices made by the particular manufacturer. Similarly, an oxygen saturation prediction model can be trained to be specific to a particular model of patient monitoring devices using training data such as PPG signals and oxygen saturation levels received and/or determined by patient monitoring devices of that particular model. As such, different models of patient monitoring devices 202 and/or patient monitoring devices 202 made by different manufacturers may select different oxygen saturation prediction models that is specific to a particular model and/or manufacturer of patient monitoring devices 202 to use in predicting the future oxygen saturation levels of patient 208.

For example, if patient monitoring device 202A and patient monitoring device 202B are the same model of patient monitoring devices made by the same manufacturer, both patient monitoring device 202A and patient monitoring device 202B may use the same oxygen saturation prediction model, such as oxygen saturation prediction model 226A, that is trained to be specific to the particular model and manufacturer of patient monitoring device 202A and patient monitoring device 202B, to predict the future oxygen saturation levels of patient 208A and patient 208B. In another example, if patient monitoring device 202A and patient monitoring device 202B are patient monitoring devices made by different manufacturers, patient monitoring device 202A may use an oxygen saturation prediction model, such as oxygen saturation prediction model 226A, that is specific to the manufacturer of patient monitoring device 202A to predict the future oxygen saturation levels of patient 208A. Similarly, patient monitoring device 202B may use an oxygen saturation prediction model, such as oxygen saturation prediction model 226B, that is specific to the manufacturer of patient monitoring device 202B to predict the future oxygen saturation levels of patient 208B.

In some examples, oxygen saturation prediction models 226 include one or more oxygen saturation prediction models that are trained to predict the future oxygen saturation levels of particular patient demographics. For example, an oxygen saturation prediction model may be specific to patients in a particular age range (e.g., patients between 65 years of age and 75 years of age), patients having a specific comorbidity, patients in a particular geographical region, patients in a specific medical facility (e.g., hospital, hospital unit, clinic), and the like. That is, an oxygen saturation prediction model may be trained to be used to predict the future oxygen saturation levels of patients in a particular patient demographic, such as by training the oxygen saturation prediction model using training data such as PPG signals and oxygen saturation levels received from and/or determined for patients belonging to the particular patient demographic.

A patient monitoring device 202 may select, for a patient, an oxygen saturation prediction model that is associated with the demographic of the patient for use to predict the future oxygen saturation levels of the patient. For example, if patient 208A is a patient who is 50 years old and has a history of high blood pressure, patient monitoring device 202A may select, for patient 208A, an oxygen saturation prediction model, such as oxygen saturation prediction model 226A, that has been trained to be used for patients that are between the ages of 45 and 55 and has a history of high blood pressure, for use to predict the future oxygen saturation levels of patient 208A.

In some examples, oxygen saturation prediction models 226 may include one or more patient-specific oxygen saturation prediction models. That is, an oxygen saturation prediction model may be trained to predict the future oxygen saturation levels of a particular patient, such as by training the oxygen saturation prediction model using training data such as PPG signals and oxygen saturation levels received from and/or determined for the particular patient.

A patient-specific oxygen saturation prediction model maybe stored in or otherwise indicated by the electronic medical records of the corresponding patients. For example, the electronic medical record of a patient may store model weights and other model information that may be used to generate an oxygen saturation prediction model that is specific to the patient. In some examples, the electronic medical record of a patient may store model weights by storing a difference between the model weights for the oxygen saturation prediction model that is specific to the patient and the baseline weights of a baseline oxygen saturation prediction model. In some examples, the electronic medical record may store such model weights and other model information that may be used to generate an oxygen saturation prediction model that is specific to the patient in encrypted form.

Different patient monitoring devices in different hospitals and clinics may therefore be able to access the patient-specific oxygen saturation prediction model for a particular patient from the patient's electronic medical records, thereby enabling usage of such a patient-specific oxygen saturation prediction model to treat the patient at different locations using different patient monitoring devices. For example, patient monitoring device 202A that monitors the oxygen saturation level of patient 208A may generate, based on the model data stored in the electronic medical record of patient 208A, oxygen saturation prediction model 226A that is specific to patient 208A. Patient monitoring device 202A may therefore use oxygen saturation prediction model 226A to predict the future oxygen saturation levels of patient 208A.

In some examples, each of patient monitoring devices 202 is configured to perform continuous or near continuous monitoring of the oxygen saturation level of one or more patients and to, in response to the oxygen saturation level of a corresponding patient decreasing to reach a desaturation threshold, use one or more of oxygen saturation prediction models 226 to predict whether the oxygen saturation level of the corresponding patient will increase back above the desaturation threshold within a predefined time period (e.g., less than 2 seconds, 5 seconds, 10 seconds, 15 seconds, and the like from a desaturation time at which the oxygen saturation level reached the desaturation threshold). For example, patient monitoring device 202A is configured to monitor the oxygen saturation levels of patient 208A and to, in response to determining that the oxygen saturation level of patient 208A has decreased to reach a desaturation threshold, use oxygen saturation prediction model 226A to predict whether the oxygen saturation level of patient 208A will increase back above the desaturation threshold within a predefined time period.

Although illustrated in FIG. 2 as executing at oxygen saturation prediction system 220, each of patient monitoring devices 202 may, in some examples, execute one or more of oxygen saturation prediction models 226 to predict whether the oxygen saturation level of a corresponding patient will increase back above the desaturation threshold within a predefined time period. For example, patient monitoring device 202A may be configured to execute oxygen saturation prediction model 226A to predict, in response to the oxygen saturation level of patient 208A decreasing to reach a desaturation threshold, whether the oxygen saturation level of patient 208A will increase back above the desaturation threshold within a predefined time period, such as within 15 seconds. The patient monitoring device 202A may continuously collect data during the predefined time period (e.g., the 15 seconds) and use (e.g., analyze) the collected data to determine whether the prediction was accurate. As such, in an embodiment, the oxygen saturation prediction model 226A may adjust itself in real-time based on the collected data.

Further, patient monitoring device 202A may be configured to use data such as the oxygen saturation levels of patient 208A, a heart rate (e.g., a pulse rate) of the patient 208A, the blood pressure of patient 208A, demographics (e.g., characteristics) of the patient 208A, and/or one or more metrics derived from the PPG signals of patient 208A (which may include the heart rate of the patient) as input to oxygen saturation prediction model 226A, and patient monitoring device 202A may be configured to execute oxygen saturation prediction model 226A to predict, based on the inputted data, whether the oxygen saturation level of patient 208A will increase back above the desaturation threshold within a predefined time period.

In some examples, one or more of oxygen saturation prediction models 226 may execute at oxygen saturation prediction system 220 to predict whether the oxygen saturation level of a patient will increase back above the desaturation threshold within a predefined time period. In these examples, patient monitoring device 202A may be configured to, in response to the oxygen saturation level of patient 208A decreasing to reach a desaturation threshold, send, to oxygen saturation prediction system 220, a request to predict whether the oxygen saturation level of patient 208A will increase back above the desaturation threshold within a predefined time period. The request may indicate the oxygen saturation levels of patient 208A, the blood pressure of patient 208A, one or more metrics derived from the PPG signals of patient 208A, and/or other inputs disclosed herein. The request may also indicate one or more of oxygen saturation prediction models 226, such as oxygen saturation prediction model 226A, to be used to predict whether the oxygen saturation level of patient 208A will increase back above the desaturation threshold within a predefined time period.

Oxygen saturation prediction system 220 may be configured to, in response to receiving the request, execute the one or more of oxygen saturation prediction models 226 indicated by the request, such as oxygen saturation prediction model 226A, to use the oxygen saturation levels of patient 208A, the blood pressure of patient 208A, and/or one or more metrics derived from the PPG signals of patient 208A indicated by the request as input data to predict, based on the input data, whether the oxygen saturation level of patient 208A will increase back above the desaturation threshold within a predefined time period. Oxygen saturation prediction system 220 may therefore be configured to send, to patient monitoring device 202A, an indication of whether the oxygen saturation level of patient 208A is predicted to increase back above the desaturation threshold within a predefined time period, and patient monitoring device 202A may be configured to receive, from oxygen saturation prediction system 220, the indication of whether the oxygen saturation level of patient 208A is predicted to increase back above the desaturation threshold within a predefined time period.

Each of patient monitoring devices 202 may be configured to, in response to one or more of oxygen saturation prediction models 226 predicting that the oxygen saturation level of a corresponding patient will not increase above the desaturation threshold by the end of a corresponding prediction period, output a notification indicative of a corresponding patient experiencing an oxygen desaturation event. Such a notification may be in the form of an audible notification, a visual notification, and the like. In this way, the patient monitoring devices 202 may make the prediction prior to providing the notification, but the predication may be made quickly (e.g., within milliseconds or seconds; only as long as needed to process the inputs to make the prediction) so that the notification may be provided within a clinically acceptable or reasonable time of the oxygen desaturation event.

In examples described herein, each of patient monitoring devices 202 is configured to, in response to one or more of oxygen saturation prediction models 226 predicting that the oxygen saturation level of a corresponding patient will increase above the desaturation threshold by the end of a corresponding prediction period (e.g., a predefined time period), refrain from outputting a notification indicative of the corresponding patient experiencing an oxygen desaturation event. Instead, each of patient monitoring devices 202 is configured to determine, at the end of the corresponding prediction period, whether to output a notification indicative of an oxygen desaturation event. For example, if patient monitoring device 202A uses oxygen saturation prediction model 226A to predict that the oxygen saturation level of patient 208A will increase above the desaturation threshold by the end of a predefined time period, then patient monitoring device 202A may be configured to, at the end of the predefined time period, determine whether the oxygen saturation level of patient 208A has actually increased above the desaturation threshold.

If patient monitoring device 202A determines that the oxygen saturation level of patient 208A is above the desaturation threshold at the end of the predefined time period, then patient monitoring device 202A may be configured to continue to refrain from outputting a notification indicative of patient 208A experiencing an oxygen desaturation event. Conversely, if patient monitoring device 202A determines that the oxygen saturation level of patient 208A is not above the desaturation threshold at the end of the predefined time period (which as noted above may be less than 2 seconds, 5 seconds, 10 seconds, or 15 seconds), then patient monitoring device 202A may be configured to output a notification indicative of patient 208A experiencing an oxygen desaturation event. In this way, patient monitoring devices 202 may be configured to reduce nuisance alerts indicative of trivial oxygen desaturation events, while still providing timely indications of more non-trivial desaturation events.

Computing system 210 is configured to execute model accuracy module 212 to track the prediction performance of one or more of oxygen saturation prediction models 226 across a plurality of predictions to determine whether the prediction performance of the one or more of oxygen saturation prediction models 226 meets a performance metric across the plurality of predictions. The prediction performance of oxygen saturation prediction models 226 may vary across a population of patients. For example, the prediction performance of oxygen saturation prediction models may vary for different patients with different skin pigmentations, for different patients of different age ranges, for different patients with different confounding comorbidities and the like. The prediction performance of oxygen saturation prediction models may also change over time, such as due to changes in the patient population, changes in the standard procedures of a hospital, changes in medications, and the like.

Because patient monitoring devices 202 monitor the actual oxygen saturation levels of patients 208, including monitoring the oxygen saturation levels of patients 208 after a prediction has been made, each patient monitoring device that uses an oxygen saturation prediction model to predict whether the oxygen saturation level of a patient will increase above a desaturation threshold by the end of a predetermined time period is able to determine the actual oxygen saturation level of the patient at the end of the predetermined time period, also referred to as a ground truth (e.g., a truth reference value) of (e.g., associated with) the prediction. As such, a patient monitoring device is able to determine (e.g., in real-time; during a monitoring session), for each prediction made by an oxygen saturation prediction model, the accuracy of the prediction by monitoring the ground truth (e.g., the actual oxygen saturation level of a patient) and by comparing the ground truth to the prediction made to identify whether the ground truth and the prediction are consistent.

Each time a patient monitoring device uses an oxygen saturation prediction model to predict whether the oxygen saturation level of a patient will increase above a desaturation threshold by the end of a predetermined time period, the patient monitoring device may send, to oxygen saturation prediction system 220, prediction data associated with the prediction. The prediction data may include the input data used by the oxygen saturation prediction model to make the prediction, the prediction made by the oxygen saturation prediction model, an indication of whether the prediction was accurate, the confidence of the prediction (e.g., a confidence score associated with the prediction as determined by the oxygen saturation prediction model), the actual oxygen saturation level of the patient at the end of the predetermined time period (e.g., the ground truth), information regarding the patient associated with the prediction, and the like.

As described above, because a patient monitoring device continues to monitor the oxygen saturation levels of patients 208 after a prediction has been made, each patient monitoring device that uses an oxygen saturation prediction model to predict whether the oxygen saturation level of a patient will increase above a desaturation threshold by the end of a predetermined time period is able to determine the actual oxygen saturation level of the patient at the end of the predetermined time period. As such, oxygen saturation prediction system 220 may be able to receive, for each prediction, the ground truth of the prediction without having to perform any manual labeling of the prediction data, which may enable oxygen saturation prediction system 220 to efficiently and automatically perform, in real time, one or more corrective actions regarding an oxygen saturation prediction model based on the prediction performance of the oxygen saturation prediction model, as described below.

Oxygen saturation prediction system 220 may execute model accuracy module 212 to receive and store such prediction data. By receiving and storing such prediction data, model accuracy module 212 may be able to use that data to assess the prediction performance of an oxygen saturation prediction model over time, such as by determining whether the prediction performance of the oxygen saturation prediction model meets a performance metric. For example, model accuracy module 212 may be able to determine whether the prediction performance of an oxygen saturation prediction model meets the performance metric based on data associated with accuracy, sensitivity, specificity, and the like, which may be included in the prediction data. Model accuracy module 212 may also be able to determine the effectiveness of an oxygen saturation prediction model in terms of how many alarms (e.g., notifications of desaturation events) were suppressed and/or the overall reduction in alarm burden based on the predictions made using the oxygen saturation prediction model.

In some examples, the prediction performance of an oxygen saturation prediction model may be an accuracy level of the oxygen saturation prediction model, which may be the percentage of accurate predictions made by the oxygen saturation prediction model across the plurality of predictions, and the performance metric may be associated with a minimum accuracy threshold. In some embodiments, the performance metric may also be associated with an accuracy level or value, a sensitivity level or value (e.g., a true positive rate), and a specificity level or value (e.g., a true negative rate). The accuracy level may be based on a threshold of accuracy (e.g., the minimum accuracy level) that the oxygen saturation model is expected to maintain to be operable in intended applications. The sensitivity level may be associated with a probability of a positive test result, which is conditioned on the patient truly being positive. Moreover, the specificity level may be associated with a probability of a negative test result, which is conditioned on the patient truly being negative. It should be appreciated that the performance metric may refer to or include multiple performance metrics (e.g., separate thresholds for accuracy, sensitivity, and/or specificity) implemented or considered to evaluate the prediction performance of an oxygen saturation prediction model.

For example, computing system 210 may be configured to execute model accuracy module 212 to determine whether the prediction performance of the oxygen saturation prediction model meets a performance metric by at least determining whether the accuracy level of the oxygen saturation prediction model is at or above a minimum accuracy threshold. If model accuracy module 212 determines that the accuracy level of the oxygen saturation prediction model is at or above the minimum accuracy threshold, then model accuracy module 212 determines that the prediction performance of the oxygen saturation prediction model meets the performance metric. If model accuracy module 212 determines that the accuracy level of the oxygen saturation prediction model is below the minimum accuracy threshold, then model accuracy module 212 determines that the prediction performance of the oxygen saturation prediction model does not meet the performance metric. It should be appreciated that similar techniques may be implemented with sensitivity and/or specificity, in addition to or as an alternative to accuracy. In certain embodiments, correction actions may be taken in response to the prediction performance failing to meet one or more of the performance metrics (e.g., one of accuracy, sensitivity, or specificity) or failing to meet two or more of the performance metrics (e.g., two or all of accuracy, sensitivity, or specificity). It should be appreciated that additional or alternative performance measures (e.g., prediction performance measures) and performance metrics may be implemented.

Computing system 210 is configured to execute model accuracy module 212 to, in response to determining that the prediction performance of a particular oxygen saturation prediction model 226 does not meet the performance metric, perform one or more corrective actions associated with the particular oxygen saturation prediction model 226. For example, if computing system 210 determines that the prediction performance of oxygen saturation prediction model 226A does not meet the performance metric, then computing system 210 may execute model accuracy module 212 to perform one or more corrective actions associated with oxygen saturation prediction model 226A.

In some examples, the one or more corrective actions associated with an oxygen saturation prediction model include disabling the oxygen saturation prediction model. For example, if computing system 210 determines that the accuracy level of oxygen saturation prediction model 226A is below a minimum accuracy threshold, then computing system 210 may be configured to execute model accuracy module 212 to perform a corrective action. For example, the model accuracy module 212 may be executed to disable (e.g., turn off) the oxygen saturation prediction model 226A. As such, the one or more patient monitoring devices 202 may revert to a default state (e.g., reinstate baseline functionality). In an embodiment, the clinician may override the model accuracy module 212 to maintain operation of the oxygen saturation prediction model 226A.

As another example, the model accuracy module 212 may be executed to update (e.g., re-train) the oxygen saturation prediction model 226A. Indeed, the oxygen saturation prediction model 226A may be updated based on data that caused the oxygen saturation prediction model 226A to perform inaccurately. As yet another example, data associated with variations in performance may be recorded and analyzed to provide insight into the performance of the oxygen saturation prediction model 226A under various conditions. Indeed, the data associated with the variations in performance may be provided to a manufacturer for incorporation into future datasets at a subsequent time. As yet another example, the model accuracy module 212 may be executed to select a different model of the one or more oxygen saturation prediction models 226. That is, an oxygen saturation prediction model of the one or more oxygen saturation prediction models 226 with a higher (e.g., better) performance than the oxygen saturation prediction model 226A may be selected and implemented.

In cases in which model accuracy module 212 granularly tracks the prediction performance of oxygen saturation prediction model 226A for different categories of use cases, computing system 210 is configured to execute model accuracy module 212 to, in response to the prediction performance of an oxygen saturation prediction model for a particular use case not meeting the performance metric, disable usage of oxygen saturation prediction model 226A for the particular use case. For example, if model accuracy module 212 determines that prediction performance of an oxygen saturation prediction model for a particular patient demographic does not meet the performance metric, then computing system 210 is configured to execute model accuracy module 212 to disable usage of oxygen saturation prediction model 226A of patients in that patient demographic.

To disable oxygen saturation prediction model 226A, computing system 210 may be configured to execute model accuracy module 212 to send, to oxygen saturation prediction system 220 and/or to each patient monitoring device using oxygen saturation prediction model 226A, a request to disable usage of oxygen saturation prediction model 226A, thereby enabling oxygen saturation prediction system 220 and/or each of the patient monitoring devices to disable usage of oxygen saturation prediction model 226A. For example, if patient monitoring device 202A is using oxygen saturation prediction model 226A to predict the future oxygen saturation levels of patient 208A, patient monitoring device 202A may be configured to receive, from computing system 210, a request to disable usage of oxygen saturation prediction model 226A. Patient monitoring device 202A may, in response to receiving the request, disable usage of oxygen saturation prediction model 226A. In some examples, patient monitoring device 202A may, in response to receiving the request, output a notification indicative of receiving the request to disable usage of oxygen saturation prediction model 226A, and a user may be able to interact with patient monitoring device 202A to override the disabling of oxygen saturation prediction model 226A.

In some examples, if patient monitoring device 202A is using oxygen saturation prediction model 226A to predict the future oxygen saturation levels of patient 208A, then patient monitoring device 202A may, after disabling usage of oxygen saturation prediction model 226A, continue to monitor the oxygen saturation levels of patient 208A. Patient monitoring device 202A may, in response to determining that the blood oxygen saturation level of patient 208A decreased to reach a desaturation threshold, immediately begin outputting a notification indicative of a desaturation event instead of using an oxygen saturation prediction model to predict whether the blood oxygen saturation level of patient 208A will increase above the desaturation threshold at the end of a predefined time period.

An oxygen saturation prediction model that has been disabled may continue to execute at oxygen saturation prediction system 220 or at one or more of patient monitoring devices 202 to predict the future oxygen saturation levels of one or more of patients 208 even if the predictions made by the disabled oxygen saturation prediction model are not used by one or more of patient monitoring devices 202 to take an action, such as disabling notifications indicative of desaturation events. Model accuracy module 212 may be configured to continue to track the prediction performance of the disabled oxygen saturation prediction model and may, in response to determining that the prediction performance of the disabled oxygen saturation prediction model increases to meet the performance metric, reenable the disabled oxygen saturation prediction model.

For example, while oxygen saturation prediction model 226A is disabled, patient monitoring device 202A and/or oxygen saturation prediction system 220 may be configured to continue to execute oxygen saturation prediction model 226A to predict, each time the oxygen saturation level of patient 208A decreases to reach a desaturation threshold, whether the oxygen saturation level of patient 208AB will increase above the desaturation threshold by the end of a predetermined period. Such predictions made by oxygen saturation prediction model 226A while oxygen saturation prediction model 226A is disabled may be referred to as background predictions. Computing system 210 may be configured to execute model accuracy module 212 to track the prediction performance of such background predictions made by oxygen saturation prediction model 226A while oxygen saturation prediction model 226A is disabled.

Computing system 210 may be configured to execute model accuracy module 212 to, in response to determining that the prediction performance of the background predictions made by oxygen saturation prediction model 226A meets the performance metric, reenable oxygen saturation prediction model 226A, such as by sending, to one or more of patient monitoring devices 202, a request to reenable oxygen saturation prediction model 226A. Once oxygen saturation prediction model 226A has been reenabled, patient monitoring device 202A may use oxygen saturation prediction model 226A to predict, in response to the oxygen saturation level of patient 208A decreasing to reach a desaturation threshold, whether the oxygen saturation level of patient 208A will increase above the desaturation threshold by the end of a predetermined period. If oxygen saturation prediction model 226A predicts that the oxygen saturation level of patient 208A will increase above the desaturation threshold by the end of a predetermined period, oxygen saturation prediction system 220 may refrain from outputting a notification indicative of a desaturation event.

As noted above, if computing system 210 determines that the prediction performance of a particular oxygen saturation prediction model 226 does not meet a performance metric, then computing system 210 performs one or more corrective actions. The corrective actions may enable patient monitoring devices 202 to more accurately predict the future oxygen saturation levels of patients. In some examples, the one or more corrective actions include causing one or more of patient monitoring devices 202 to stop using an oxygen saturation prediction model 226 having a prediction performance that does not meet the performance metric and to instead use a different oxygen saturation prediction model 226. For example, computing system 210 may be configured to execute model accuracy module 212 to, in response to determining that the prediction performance of oxygen saturation prediction model 226A does not meet the performance metric, cause one or more of patient monitoring devices 202 to stop using oxygen saturation prediction model 226A for prediction and to instead use a different oxygen saturation prediction model, such as oxygen saturation prediction model 226B, to predict the future oxygen saturation levels of patients.

In some examples, computing system 210 may execute model accuracy module 212 to select another oxygen saturation prediction model 226 that one or more of patient monitoring devices 202 may switch to using based on the prediction performance of that oxygen saturation prediction model. When patient monitoring device 202A uses a single oxygen saturation prediction model, such as oxygen saturation prediction model 226A, to predict the future oxygen saturation levels of patient 208A, patient monitoring device 202A and/or oxygen saturation prediction system 220 may execute one or more other oxygen saturation prediction models, such as one or more of oxygen saturation prediction models 226B-226P, in the background that may, each time oxygen saturation prediction model 226A makes a prediction of the future oxygen saturation levels of patient 208A, also each make a prediction of the future oxygen saturation levels of patient 208A. Such one or more of oxygen saturation prediction models 226B-226P that execute in the background to predict the future oxygen saturation levels of patient 208A may be models trained to be specific to patient 208A, one or more patient demographics to which patient 208A belongs, a clinic in which patient 208A is located, and the like.

Computing system 210 may be configured to execute model accuracy module 212 to also track the prediction performance of each of the one or more of patient monitoring devices 202 when executing in the background to predict the future oxygen saturation levels of patient 208A. Computing system 210 may therefore be configured to execute model accuracy module 212 to, in response to disabling usage of oxygen saturation prediction model 226A, select, based on the prediction performance of each of the one or more of oxygen saturation prediction models 226B-226P when executing in the background to predict the future oxygen saturation levels of patient 208A, another oxygen saturation prediction model for use by patient monitoring device 202A in place of oxygen saturation prediction model 226A.

For example, computing system 210 may be configured to execute model accuracy module 212 to select, as the replacement for oxygen saturation prediction model 226A, the oxygen saturation prediction model that has the highest prediction performance out of the one or more of oxygen saturation prediction models 226B-226P executing in the background. Computing system 210 may be configured to execute model accuracy module 212 to select the replacement for oxygen saturation prediction model 226A after the one or more of patient monitoring devices 202 have made a certain number of predictions or after a specified amount of time after disabling oxygen saturation prediction model 226A, such as after 15 minutes, 30 minutes, 45 minutes, 60 minutes, or the like.

Computing system 210 may therefore be configured to execute model accuracy module 212 to send, to patient monitoring device 202A, a request to switch to using the selected oxygen saturation prediction model to predict the future oxygen saturation levels of patient 208A. Patient monitoring device 202A may, in response to receiving the request, switch to using the oxygen saturation prediction model indicated by the request to predict the future oxygen saturation levels of patient 208A.

In some examples, switching one or more of patient monitoring devices 202 away from using an oxygen saturation prediction model having a prediction performance that does not meet the performance metric to using a different oxygen saturation prediction model includes updating the oxygen saturation prediction model and switching the one or more of patient monitoring devices 202 to using the updated oxygen saturation prediction model. Updating an oxygen saturation prediction model may enable the updated oxygen saturation prediction model to have a prediction performance that meets the performance metric. In some examples, computing system 210 updates an oxygen saturation prediction model by at least updating the model based on new training data (e.g., which may include the ground truth data). Thus, in some examples, computing system 210 updates an oxygen saturation prediction model by at least retraining an existing model using a relatively small amount of data so that it performs better on the new data.

For example, computing system 210 may be configured to, in response to determining that the prediction performance of oxygen saturation prediction model 226A is below the performance metric, execute model accuracy module 212 to update (also referred to herein as fine tune) oxygen saturation prediction model 226A, such as by sending, to training system 230 to perform fine-tuning of oxygen saturation prediction model 226A. Training system 230 may be configured to, in response to receiving the request to perform updating of oxygen saturation prediction model 226A, perform one or more machine learning techniques to update oxygen saturation prediction model 226A, which may enable patient monitoring devices 202 to adapt, in real-time, oxygen saturation prediction model 226A to more accurately make predictions of future oxygen saturation levels of patients 208 without any input from manufacturers of patient monitoring devices 202. In some examples, to update oxygen saturation prediction model 226A, training system 230 freezes one or more layers of oxygen saturation prediction model 226A and/or uses a slow training rate to re-train oxygen saturation prediction model 226A with new training data. Such training data may include data associated with incorrect predictions made by oxygen saturation prediction model 226A. For example, the training data may include, for each of the plurality of incorrect predictions made by oxygen saturation prediction model 226A, the input data used by oxygen saturation prediction model 226A to make the corresponding incorrect prediction and the actual blood oxygen saturation level at the end of a predefined period.

In some examples, training system 230 may be configured to perform fine tuning of oxygen saturation prediction model 226A to re-train oxygen saturation prediction model 226A to be specific to a particular domain, such as a particular patient, a particular patient demographic, a particular medical facility, and the like. Training system 230 may re-train a generalized oxygen saturation prediction model to be specific to a particular domain, or may re-train an oxygen saturation prediction model that is specific to a particular domain to be specific to a different domain.

Training system 230 may be configured to re-train an oxygen saturation prediction model, such as oxygen saturation prediction model 226A, to be specific to a particular domain by at least re-training oxygen saturation prediction model 226A using training data associated with the particular domain. For example, to re-train oxygen saturation prediction model 226A to be specific to patient 208A, training system 230 may be configured to use training data that includes data associated with a plurality of incorrect predictions made by oxygen saturation prediction model 226A regarding the oxygen saturation levels of patient 208A. In another example, to re-train oxygen saturation prediction model 226A to be specific to a particular patient demographic, the training data may include data associated with a plurality of incorrect predictions made by oxygen saturation prediction model 226A regarding the oxygen saturation levels of patients in the particular patient demographic.

Training system 230 may be configured to update an oxygen saturation prediction model, such as oxygen saturation prediction model 226A, to generate an updated oxygen saturation prediction model, such as updated oxygen saturation prediction model 226B. In some examples, computing system 210 and/or patient monitor device 202A may determine whether the updated oxygen saturation prediction model meets a performance parameter before deploying the oxygen saturation prediction model for use in monitoring a patient. For example, once training system 230 has finished updating an oxygen saturation prediction model 226A to generate updated oxygen saturation prediction model 226B, updated oxygen saturation prediction model 226B may execute in the background of oxygen saturation prediction system 220 and/or patient monitoring device 202A to predict the future oxygen saturation levels of patients, such as patient 208A. Computing system 210 may execute model accuracy module 212 to track the prediction performance of the updated oxygen saturation prediction model 226B and may, in response to determining that the prediction performance of the updated oxygen saturation prediction model 226B meets a specified performance metric, such as by having an accuracy level that meets the minimum accuracy threshold, enable the updated oxygen saturation prediction model 226B to be used by patient monitoring devices 202 to predict the future oxygen saturation levels of patients 208.

After patient monitoring device 202A switches to using updated oxygen saturation prediction model 226B, patient monitoring device 202A may output, at a display device, an indication that patient monitoring device 202A is now using the updated oxygen saturation prediction model 226B. For example, patient monitoring device 202A may output the message “AI Patient Tailored Monitoring Now in Operation” or “AI Mode Now Running” to indicate that patient monitoring device 202A is now using an updated oxygen saturation prediction model 226B that has been updated to more accurately predict the future oxygen saturation levels of patients.

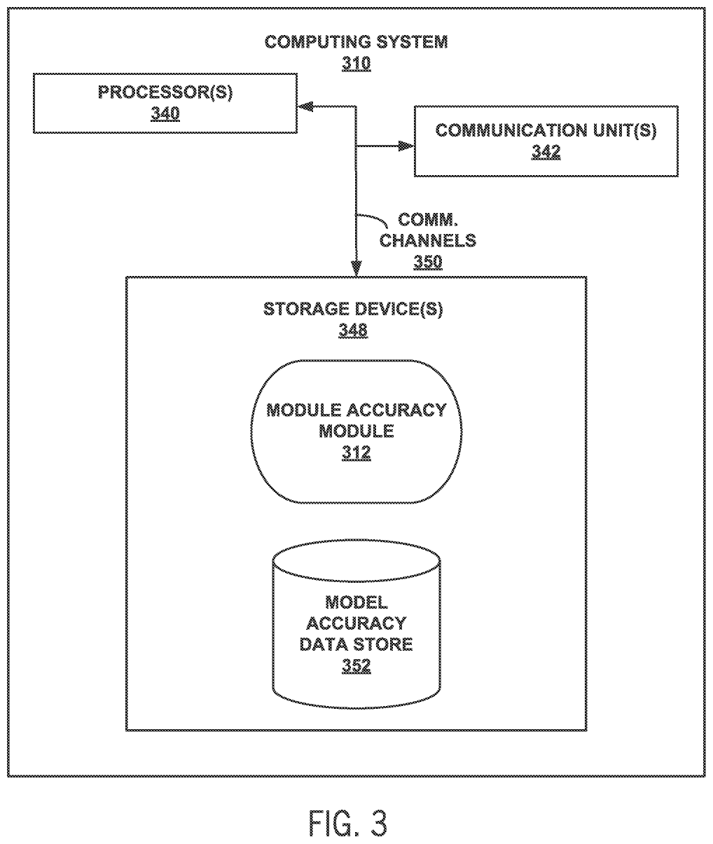

FIG. 3 is a block diagram illustrating an example computing system, in accordance with one or more aspects of the present disclosure. Computing system 310 of FIG. 3 is described below as an example of computing system 210 of FIG. 2. FIG. 3 illustrates only one particular example of computing system 310, and many other examples of computing system 310 may be used in other instances and may include a subset of the components included in example computing system 310 or may include additional components not shown in FIG. 3. For example, computing system 310 may comprise a cluster of servers, and each of the servers comprising the cluster of servers making up computing system 310 may include all, or some, of the components described herein in FIG. 3, to perform the techniques disclosed herein.

As shown in the example of FIG. 3, computing system 310 includes one or more processors 340, each including processing circuitry, one or more communication units 342, and one or more storage devices 348. Storage devices 348 include model accuracy module 312 and model accuracy data store 352.

One or more processors 340 may implement functionality and/or execute instructions associated with computing system 310. Examples of one or more processors 340 include application processors, display controllers, auxiliary processors, one or more sensor hubs, and any other hardware configure to function as a processor, a processing unit, or a processing device. Model accuracy module 312 may be operable by one or more processors 340 to perform various actions, operations, or functions of computing system 310. For example, one or more processors 340 of computing system 310 may retrieve and execute instructions stored by one or more storage devices 348 that cause one or more processors 340 to perform the operations of model accuracy module 312. The instructions, when executed by one or more processors 340, may cause computing system 310 to store information within one or more storage devices 348, for example, at model accuracy data store 352.

One or more communication units 342 of computing system 310 may communicate with external devices via one or more wired and/or wireless networks by transmitting and/or receiving network signals on the one or more networks. Examples of one or more communication units 342 include a network interface card (e.g., such as an Ethernet card), an optical transceiver, a radio frequency transceiver, a global positioning satellite (GPS) receiver, or any other type of device that can send and/or receive information. Other examples of one or more communication units 342 may include short wave radios, cellular data radios, wireless network radios, as well as universal serial bus (USB) controllers.

Communication channels 350 may interconnect each of the components 340, 342, and 348 for inter-component communications (physically, communicatively, and/or operatively). In some examples, communication channels 350 may include a system bus, a network connection, an inter-process communication data structure, or any other method for communicating data.

One or more storage devices 348 within computing system 310 may store information for processing during operation of computing system 310 (e.g., computing system 310 may store data accessed by model accuracy module 312 and model accuracy data store 352 during execution at computing system 310). In some examples, one or more storage devices 348 is a temporary memory, meaning that a primary purpose of one or more storage devices 348 is not long-term storage. In this example, one or more storage devices 348 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

In some examples, one or more storage devices 348 may also include one or more computer-readable storage media. One or more storage devices 348, in some examples, include one or more non-transitory computer-readable storage mediums. One or more storage devices 348 may be configured to store larger amounts of information than typically stored by volatile memory. One or more storage devices 348 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. One or more storage devices 348 may store program instructions and/or information (e.g., data) associated with model accuracy module 312 and model accuracy data store 352. Storage devices 348 may include a memory configured to store data or other information associated with model accuracy module 312 and model accuracy data store 352.

Model accuracy data store 352 may be any data storage mechanism for storing structured and/or unstructured data, and may be configured to store any data related to the accuracy of oxygen saturation prediction models determined or generated by model accuracy module 312. For example, model accuracy data store 352 may store, for each prediction made by an oxygen saturation prediction model tracked by model accuracy module 312, the input data used by the oxygen saturation prediction model to make the prediction, the prediction made by the oxygen saturation prediction model, an indication of whether the prediction was accurate, and the ground truth of the prediction. Examples of model accuracy data store 352 may include a database, a schema, a key-value store, and the like.

One or more processors 340 are configured to execute model accuracy module 312 to track the predictions made by one or more oxygen saturation prediction models (e.g., one or more of oxygen saturation prediction models 226 shown in FIG. 2) as one or more patient monitoring devices (e.g., one or more of patient monitoring devices 202) use the one or more oxygen saturation prediction models to predict the future oxygen saturation levels of one or more patients. For example, one or more processors 340 may be configured to execute model accuracy module 312 to track the predictions made by an oxygen saturation prediction model over a plurality of predictions to determine a prediction performance of the predictions made by the oxygen saturation prediction model. One or more processors 340 may be configured to execute model accuracy module to track the prediction performance of an oxygen saturation prediction model across a specific number of predictions, such as 200 predictions, 300 predictions, 500 predictions, and the like, or to track the prediction performance of an oxygen saturation prediction models over a specified time period, such as over thirty minutes, over one hour, over six hours, over twelve hours, over twenty four hours, over a week, and the like.

To track the predictions made by the oxygen saturation prediction model, one or more processors 340 may be configured to execute model accuracy module 312 to track, for each prediction made by the oxygen saturation prediction model across a plurality of predictions, whether the prediction made by the oxygen saturation prediction model was accurate. Specifically, one or more processors 340 may be configured to execute model accuracy module 312 to track, for each prediction made by an oxygen saturation prediction model across a plurality of predictions, prediction data associated with the corresponding prediction, such as the input data used by the oxygen saturation prediction model to make the corresponding prediction, the corresponding prediction made by the oxygen saturation prediction model, whether the corresponding prediction was correct (i.e., whether the corresponding prediction was consistent with the ground truth), and the ground truth of the corresponding prediction (e.g., the actual oxygen saturation level of the patient at the end of the predefined time period). One or more processors 340 may be configured to execute model accuracy module 312 to communicate with an oxygen saturation prediction system, such as oxygen saturation prediction system 220 of FIG. 2, and/or one or more patient monitoring devices, such as one or more of patient monitoring devices 202 of FIG. 2, to receive, for each prediction made by the oxygen saturation prediction model, the prediction data associated with the corresponding prediction, and may store the prediction data for each corresponding prediction in model accuracy data store 352.

Computing system 310 may be configured to execute model accuracy module 312 to determine, based on tracking the predictions made by the oxygen saturation prediction model, the prediction performance of the oxygen saturation prediction model and to determine whether the prediction performance of the oxygen saturation prediction model meets a performance metric. For example, one or more processors 340 may be configured to execute model accuracy module 312 to access the prediction data of the predictions made by an oxygen saturation prediction model tracked by model accuracy module 312 stored in model accuracy data store 352. Computing system 310 may therefore be configured to execute model accuracy module 312 to determine, based on the prediction data, the prediction performance of the oxygen saturation prediction model and to determine whether the prediction performance of the oxygen saturation prediction model meets a performance metric.

In some examples, the prediction performance of an oxygen saturation prediction model may be an accuracy level of the oxygen saturation prediction model, which may be the percentage of accurate predictions made by the oxygen saturation prediction model across the plurality of predictions, and the prediction metric may be a minimum accuracy threshold, such as 60%, 70%, 80%, 90%, 95%, 99%, and the like. One or more processors 340 may therefore be configured to execute model accuracy module 312 to determine whether the prediction performance of the oxygen saturation prediction model meets a performance metric by determining whether the accuracy level of the oxygen saturation prediction model is at or above a minimum accuracy threshold. In some examples, if model accuracy module 312 determines that the accuracy level of the oxygen saturation prediction model is at or above the minimum accuracy threshold, then model accuracy module 212 determines that the prediction performance of the oxygen saturation prediction model meets the performance metric. In addition, in some examples, if model accuracy module 312 determines that the accuracy level of the oxygen saturation prediction model is below the minimum accuracy threshold, then model accuracy module 312 determines that the prediction performance of the oxygen saturation prediction model does not meet the performance metric.

In some examples, to determine whether the prediction performance of an oxygen saturation prediction model across a plurality of predictions meets a performance metric, one or more processors 340 executes model accuracy module 312 to compare the accuracy level of an oxygen saturation prediction model across a plurality of predictions against the accuracy level of a baseline oxygen saturation prediction model. If model accuracy module 312 determines that the accuracy level of an oxygen saturation prediction model across a plurality of predictions is equal to or greater than the accuracy level of a baseline oxygen saturation prediction model, then model accuracy module 312 determines that the prediction performance of the oxygen saturation prediction model meets the performance metric.

For example, each time an oxygen saturation prediction model is used to predict whether the oxygen saturation level of a corresponding patient will increase above a desaturation threshold by the end of a corresponding predefined time period, model accuracy module 312 may be configured to also use a baseline oxygen saturation prediction model to predict, using the same input data used by the oxygen saturation prediction model, whether the oxygen saturation level of the corresponding patient will increase above a desaturation threshold by the end of the corresponding predefined time period. Model accuracy module 312 may therefore be able to track the accuracy levels of the oxygen saturation prediction model and the baseline oxygen saturation prediction model and to compare the accuracy levels of the oxygen saturation prediction model and the baseline oxygen saturation prediction model to determine whether the prediction performance of the oxygen saturation prediction model meets the performance metric.

In some examples, computing system 310 may be configured to execute model accuracy module 312 to determine, based on the prediction data of the predictions made by the oxygen saturation prediction model for one or more use cases. For example, model accuracy module 312 may be configured to determine, based on tracking the predictions made by the oxygen saturation prediction model, the accuracy level of the predictions made by the oxygen saturation prediction model for a particular patient demographic (e.g., patients within a particular age range, patients with a particular medical condition, etc.), for a particular ward in a hospital, for specific times of the day (e.g., the morning and/or the evening), and the like.

In some examples, one or more processors 340 are configured to execute model accuracy module 312 to track the prediction performance of an oxygen saturation prediction model across a population of patients, for one or more specific patient demographics, for one or more specific patients, and/or for different use cases. For example, one or more processors 340 may be configured to execute model accuracy module 312 to track the prediction performance of an oxygen saturation prediction model for one or more different patient demographics (e.g., patients within a particular age range, patients with a particular medical condition, etc.), for particular wards of a hospital, for specific times of the day (e.g., the prediction performance during the morning and the prediction performance during the evening), and the like.

One or more processors 340 may be configured to execute model accuracy module 312 to, in response to determining that the prediction performance of an oxygen saturation prediction model does not meet the performance metric, perform one or more corrective actions associated with the oxygen saturation prediction model. In some examples, one or more processors 340 are configured to execute model accuracy module 312 to perform one or more corrective actions associated with the oxygen saturation prediction model by at least logging and reporting the decrease in performance of the oxygen saturation prediction model back to the developers of the oxygen saturation prediction model, so that the developers of the oxygen saturation prediction model may be able to work to improve the accuracy of the oxygen saturation prediction model. For example, one or more processors 340 may be configured to execute model accuracy module 312 to report data associated with a sampling of incorrect predictions made by the oxygen saturation prediction model, such as the input data used by the oxygen saturation prediction model when making such incorrect predictions, the actual oxygen saturation levels of patients at the end of the predefined time periods (e.g., the ground truths of such predictions), and any other data that may be useful to the developers of the oxygen saturation prediction model to retrain or otherwise improve the accuracy of the oxygen saturation prediction model.

In some examples, one or more processors 340 may be configured to execute model accuracy module 312 to perform one or more corrective actions associated with the oxygen saturation prediction model by at least disabling usage of the particular oxygen saturation prediction model by one or more patient monitoring devices 202. To disable usage of the oxygen saturation prediction model, one or more processors 340 may be configured to execute model accuracy module 312 to send, to each of one or more patient monitoring devices 202 using the oxygen saturation prediction model and/or to an oxygen saturation prediction system configured to execute the oxygen saturation prediction model, a request to disable usage of the oxygen saturation prediction model.

In some examples, because model accuracy module 312 may be configured to track the prediction performance of an oxygen saturation prediction model for specific use cases, such as the prediction performance of the oxygen saturation prediction model for a specific patient, a patient demographic, a time (e.g., a specific part) of the day, and the like, if model accuracy module 312 determines that the prediction performance of an oxygen saturation prediction model for a particular use case does not meet the performance metric, model accuracy module 312 disables usage of the oxygen saturation prediction model for that particular use case. One or more processors 340 may be configured to execute model accuracy module 312 to, in response to determining that the prediction performance of an oxygen saturation prediction model for a particular use case does not meet the performance metric, determine one or more patient monitoring devices that are using the oxygen saturation prediction model for the particular use case and to send, to each of the one or more patient monitoring devices 202, a request to disable usage of the oxygen saturation prediction model for that particular use case.

In some examples, an oxygen saturation prediction model that has been disabled may continue to execute at one or more patient monitoring devices 202 to predict the future oxygen saturation levels of one or more patients even if the predictions made by the disabled oxygen saturation prediction model are not used by one or more patient monitoring devices 202. One or more processors 340 may therefore be configured to execute model accuracy module 312 to continue to track the prediction performance of a disabled oxygen saturation prediction model and may, in response to determining that the prediction performance of the disabled oxygen saturation prediction model increases to meet the performance metric, reenable the disabled oxygen saturation prediction model for use by one or more monitoring devices. For example, in response to determining that the prediction performance of the disabled oxygen saturation prediction model meets a performance metric, one or more processors 340 can send a patient monitoring device 202 an instruction to begin using the previously disabled oxygen saturation prediction model.

In some examples, one or more processors 340 are configured to execute model accuracy module 312 to perform one or more corrective actions associated with the oxygen saturation prediction model by at least switching one or more of patient monitoring devices 202 away from using an oxygen saturation prediction model having a prediction performance that does not meet the performance metric to using a different oxygen saturation prediction model. For example, one or more processors 340 can be configured to cause (e.g., by sending an instruction) a patient monitoring device 202 to stop using a first oxygen saturation prediction model having a prediction performance that does not meet the performance metric and to begin using a second oxygen saturation prediction model that is different from the first oxygen saturation prediction model. In some examples, one or more processors 340 are configured to execute model accuracy module 312 to select the second oxygen saturation prediction model.

When a patient monitoring device 202 uses a single oxygen saturation prediction model to predict the future oxygen saturation levels of a patient, one or more other oxygen saturation prediction models may execute in the background to also predict the future oxygen saturation levels of one or more patients. In some examples, one or more processors 340 are configured to execute model accuracy module 312 to also track the prediction performance of each of the one or more other oxygen saturation prediction models executing in the background. In some examples, the one or more processors 340 are configured to select the second oxygen saturation prediction model based on the prediction performance of each of the one or more other oxygen saturation prediction models executing in the background. For example, one or more processors 340 may be configured to execute model accuracy module 312 to select the oxygen saturation prediction model that has the highest prediction performance out of the one or more other oxygen saturation prediction models executing in the background. One or more processors 340 may be configured to execute model accuracy module 312 to cause one or more of patient monitoring devices 202 to switch away from using an oxygen saturation prediction model having a prediction performance that does not meet the performance metric to an oxygen saturation prediction model having the highest prediction performance out of the one or more other oxygen saturation prediction models executing in the background.

In some examples, the second oxygen saturation model is an updated version of the first oxygen saturation model. Thus, in some examples, causing one or more patient monitoring devices 202 to switch away from using an oxygen saturation prediction model having a prediction performance that does not meet the performance metric to a different oxygen saturation prediction model includes updating the oxygen saturation prediction model to generate an updated oxygen saturation prediction model and causing the one or more patient monitoring devices 202 to use the updated oxygen saturation prediction model. Fine tuning an oxygen saturation prediction model may enable the updated oxygen saturation prediction model to have a prediction performance that meets the performance metric.

For example, one or more processors 340 may be configured to execute model accuracy module 312 to, in response to determining that the prediction performance of an oxygen saturation prediction model is below the performance metric, fine tune the oxygen saturation prediction model to generate an updated oxygen saturation prediction model. In some examples, model accuracy module 312 may be configured to perform fine tuning of the oxygen saturation prediction mode, or may be configured to use a training system, such as training system 230 of FIG. 2, to perform fine tuning of the oxygen saturation prediction model.

If model accuracy module 312 is configured to use a training system to update the oxygen saturation prediction model, then the training system may perform one or more machine learning techniques to fine tune the oxygen saturation prediction model, such as by freezing one or more layers of the oxygen saturation prediction model and/or by using a slow training rate to re-train the oxygen saturation prediction model with new training data. Such training data may include data associated with incorrect predictions made by the oxygen saturation prediction model. For example, the training data may include, for each of the plurality of incorrect predictions made by the oxygen saturation prediction model, the input data used by the oxygen saturation prediction model to make the corresponding incorrect prediction and the actual blood oxygen saturation level at the end of a predefined period.

In some examples, the training system may fine tune the oxygen saturation prediction model by re-training the oxygen saturation prediction model to be specific to a particular domain, such as a particular patient, a particular patient demographic, a particular medical facility, and the like. In some examples, the training system may re-train a generalized oxygen saturation prediction model to be specific to a particular domain, or may re-train an oxygen saturation prediction model that is specific to a particular domain to be specific to a different domain.

Once an oxygen saturation prediction model has been fined tuned to result in an updated oxygen saturation prediction model, the updated oxygen saturation prediction model may execute in the background to predict the future oxygen saturation levels of patients. One or more processors 340 may be configured to execute model accuracy module 312 to track the prediction performance of the updated oxygen saturation prediction model and may, in response to determining that the prediction performance of the updated oxygen saturation prediction model meets a specified performance metric, such as by having an accuracy level that meets the minimum accuracy threshold, enable the updated oxygen saturation prediction model to be used by one or more patient monitoring devices 202 to predict the future oxygen saturation levels of one or more patients.

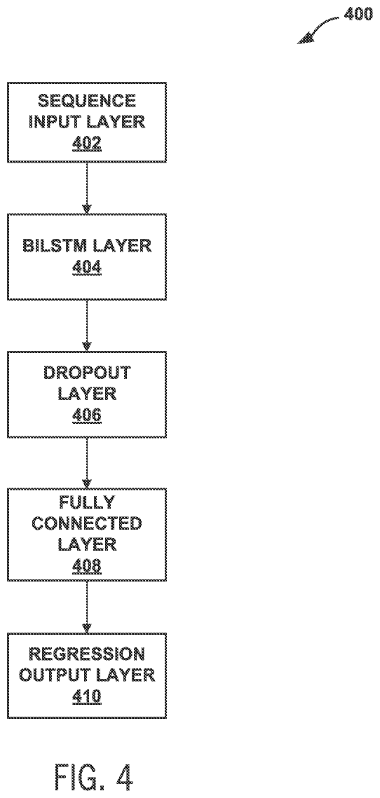

FIG. 4 illustrates an example deep learning architecture 600 of one or more of oxygen saturation prediction models 226 of FIG. 2. While deep learning architecture 400 is illustrated in FIG. 3 as being a long short-term memory (LSTM) deep learning architecture that is used to train a LSTM model, any other deep learning architectures, such as a convolutional neural network (CNN) may equally be suitable for training one or more of oxygen saturation prediction models 226.

As shown in FIG. 4, deep learning architecture 400 may include sequence input layer 402, bidirectional long short-term memory (BiLSTM) layer 404, dropout layer 406, fully connected layer 408, and regression output layer 410. Sequence input layer 402 may be connected to BILSTM layer 404. BILSTM layer 404 may be connected to dropout layer 406. Dropout layer 406 may be connected to fully connected layer 408. Fully connected layer 408 may be connected to regression output layer 410.

A sequence input layer such as sequence input layer 402 inputs sequence data to a neural network. Thus, sequence input layer 402 receives features that are used to train deep learning architecture 400. To train an oxygen saturation prediction model, sequence input layer 402 may receive features for a patient or for a population of patients, which include values of one or more of: the oxygen saturation levels of the population of patients, the blood pressures of the population of patients, and/or one or more metrics derived from the PPG signals of the population of patients that are determined during a time period immediately preceding the prediction point. The metrics derived from the PPG signals may include any combination of metrics, such as one or more of a skew of PPG pulses, PPG pulse amplitudes, normalized amplitudes of PPG pulses, PPG pulse maximum slope, the location of the PPG pulse maximum slope, PPG pulse maximum curvature, the location of the PPG pulse maximum curvature, or any other suitable morphological parameters derived from the PPG signals.

A BILSTM layer such as BILSTM layer 404 learns bidirectional long-term dependencies between time steps of time series or sequence data. These dependencies may be useful for the network to learn from a complete time series at each time step.

A dropout layer such as dropout layer 406 randomly sets input elements to zero with a given probability. By randomly setting input elements to zero, a dropout layer may enable elements to be ignored during the training phase. Selectively ignoring elements during the training phase may prevent over-fitting of training data.

A fully connected layer such as fully connected layer 408 multiplies the input (e.g., from dropout layer 406) by a weight matrix and then adds a bias vector. A regression output layer such as regression output layer 410 computes the half-mean-squared-error loss, or any other loss metric, for regression problems and outputs a predicted response of the trained regression network as a result of training the oxygen saturation prediction model having deep learning architecture 400.

To train an oxygen saturation prediction model having deep learning architecture 400, training system 230 (FIG. 2) may derive a set of features and associated target values and may input the features and the associated target values into the oxygen saturation prediction model to train the oxygen saturation prediction model to estimate target values based on the inputted features. For example, to train the oxygen saturation prediction model to predict the oxygen saturation level of a patient by the end of a predefined time period, training system 230 may extract features from one or more of: the history oxygen saturation levels of the patient during a time period immediately preceding the prediction point, the history of blood pressure values of the patient during the time period immediately preceding the prediction point, and/or one or more metrics derived from the PPG signals of the patient during the time period immediately preceding the prediction point, and may use such extracted features associated with target predicted oxygen saturation levels at the end of predefined time periods to train the oxygen saturation prediction model to predict the future oxygen saturation levels of the patient from such features.

While training of deep learning architecture 400 of an oxygen saturation prediction model is described herein as a regression problem for predicting a single continuous variable (i.e., the future oxygen saturation level of a patient; whether the oxygen saturation level of the patient will return above the desaturation level in the predefined time period), an oxygen saturation prediction model may not necessarily be limited to a regression model. In other examples, the training of deep learning architecture 400 of an oxygen saturation prediction model may be similarly formulated as a classification problem, such as classifying the future oxygen saturation level of a patient, or as any other suitable problem.

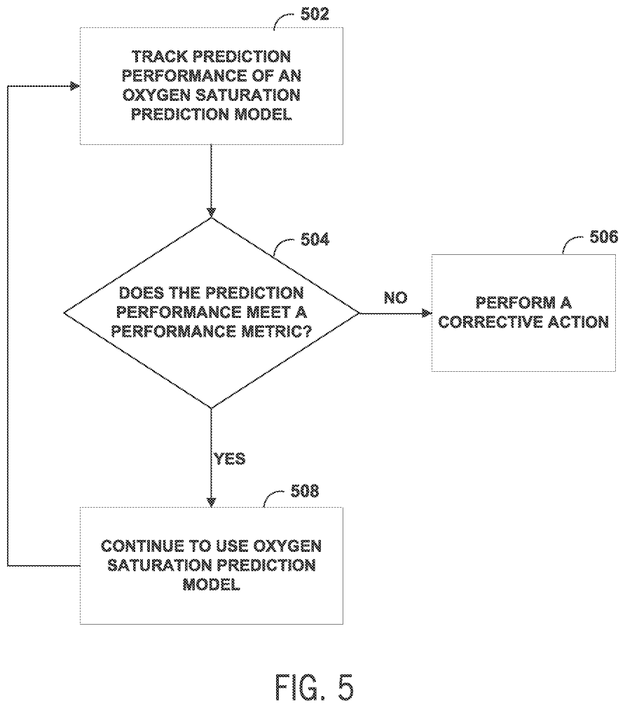

FIG. 5 is a flow diagram illustrating an example method for taking one or more corrective actions based on the predictive performance of an oxygen saturation prediction model. FIG. 5 is described with respect to computing system 210 (FIG. 2) and the function attributed to computing system 210 can be performed by processing circuitry of computing system 210 or, in other examples, processing circuitry of another device.

As shown in FIG. 5, computing system 210 may track, across a plurality of predictions, the prediction performance of an oxygen saturation prediction model used by one or more patient monitoring devices to predict the future oxygen saturation levels of patients (502). For example, computing system 210 may track the prediction performance of an oxygen saturation prediction model across a population of patients or may track the prediction performance of the oxygen saturation prediction model for a specific use case, such as for a single patient, for a particular patient demographic (e.g., age, gender, medical history, medical condition, and so on), for a particular time period (e.g., a specific part of a day, for a specific day of the week, for a specific week of a month, and so on) for a hospital, for a specific hospital ward, and the like.

Computing system 210 determines whether the prediction performance of the oxygen saturation prediction model meets a performance metric (504). For example, computing system 210 may determine the accuracy level of the oxygen saturation prediction model across the plurality of predictions, which may be in the form of the percentage of accurate predictions made by the oxygen saturation prediction model across the plurality of predictions, and may compare the accuracy level of the oxygen saturation prediction model to a minimum accuracy threshold. In some examples, if computing system 210 determines that the accuracy level of the oxygen saturation prediction model is at least equal to the minimum accuracy threshold, then computing system 210 determines that the prediction performance of the oxygen saturation prediction model meets a performance metric.

In the example shown in FIG. 5, if computing system 210 determines that the prediction performance of the oxygen saturation prediction model meets a performance metric (YES at 504), then computing system 210 continues to enable one or more patient monitoring devices 202 to continue using the oxygen saturation prediction model to predict the future oxygen saturation levels of patients (508). If computing system 210 determines that the prediction performance of the oxygen saturation prediction model does not meet a performance metric (NO at 504), then computing system 210 performs one or more corrective actions associated with the oxygen saturation prediction model (506).

As described herein, in some examples, computing system 210 performs one or more corrective actions by at least disabling the oxygen saturation prediction model, causing the one or more patient monitoring devices 202 to stop using the oxygen saturation prediction model and/or to instead use a different oxygen saturation prediction model having better prediction performance than the oxygen saturation prediction model, and/or by updating the oxygen saturation prediction model to generate an updated oxygen saturation prediction model and cause the one or more patient monitoring devices 202 to stop using the oxygen saturation prediction model to instead use the updated oxygen saturation prediction model.

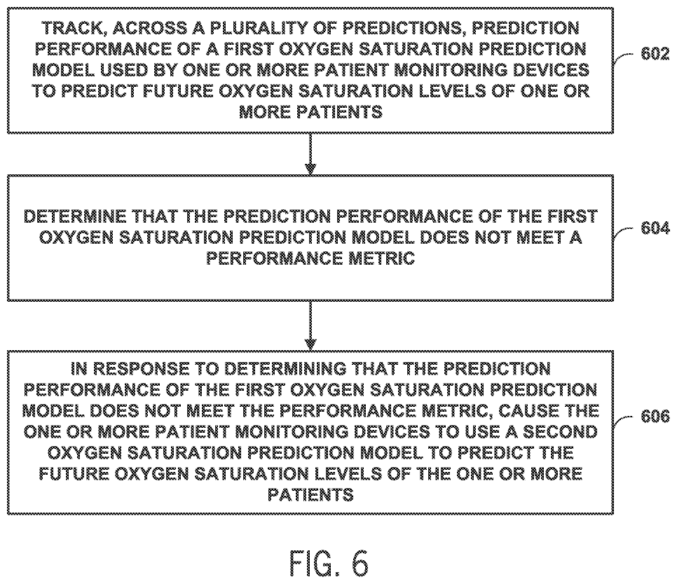

FIG. 6 is a flow diagram illustrating an example method for switching oxygen saturation models to increase the accuracy of predictions made by patient monitoring devices FIG. 6 is described with respect to computing system 210 (FIG. 2) and the function attributed to computing system 210 can be performed by processing circuitry of computing system 210 or, in other examples, processing circuitry of another device.

The technique illustrated in FIG. 6 includes computing system 210 tracking, across a plurality of predictions, prediction performance of a first oxygen saturation prediction model used by one or more patient monitoring devices to predict future oxygen saturation levels of one or more patients (602). The techniques further include the computing system 210 determining that the prediction performance of the first oxygen saturation prediction model does not meet a performance metric (604). The techniques further include the computing system 210, in response to determining that the prediction performance of the first oxygen saturation prediction model does not meet the performance metric, cause the one or more patient monitoring devices to use a second oxygen saturation prediction model to predict the future oxygen saturation levels of the one or more patients (606).

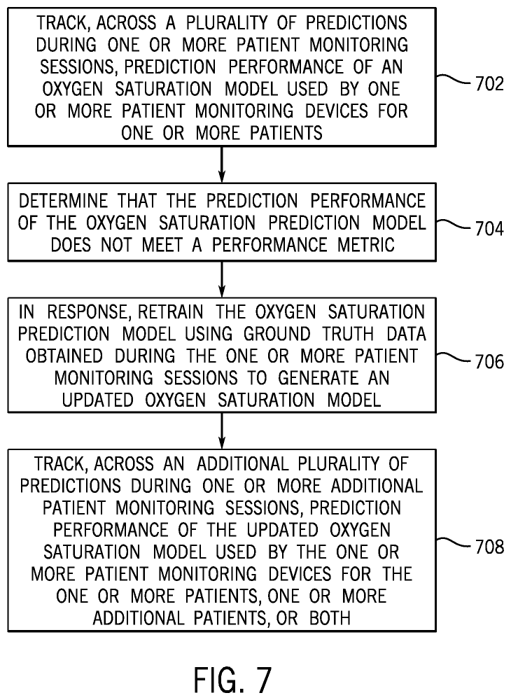

FIG. 7 is a flow diagram illustrating an example method for generating an updated oxygen saturation prediction model to increase accuracy of predictions made by patient monitoring devices. Although FIG. 7 is described with respect to computing system 210 (FIG. 2), in other examples, different processing circuitry, alone or in combination with the computing system 210, may perform any part of the technique of FIG. 7.

As shown in FIG. 7, computing system 210 may track, across a plurality of predictions during one or more patient monitoring sessions, prediction performance of an oxygen saturation model used by one or more patient monitoring devices for one or more patients (702). As described herein, the prediction performance may include a percentage of accurate predictions (e.g., of future oxygen saturation levels of patients) made by the oxygen saturation model across the plurality of predictions, and this may be carried out by comparing the predictions to actual oxygen saturation levels during the one or more patient monitoring sessions (e.g., does the prediction match or correspond to the actual oxygen saturation levels after the predefined time period; the prediction matches the actual oxygen saturation levels when, for example, it is predicted that the oxygen saturation level will not remain below the desaturation level for the predefined time period and the actual oxygen saturation did not remain below the desaturation level for the predefined time period). Indeed, the prediction performance may be associated with an efficacy of the oxygen saturation model to predict accurate (e.g., correct) outcomes based on data inputs provided to the oxygen saturation model.

The computing system 210 may then determine that the prediction performance of the oxygen saturation model does not meet a performance metric (704). The performance metric may include an accuracy level, a specificity level, and/or a sensitivity level. For example, the accuracy level may be compared to a minimum accuracy threshold to determine whether the accuracy level is less than or equal to the minimum accuracy level. In some embodiments, the computing system 210 may analyze prediction data associated with the prediction performance to determine a trend in the prediction data over time. For example, the computing system 210 may analyze the prediction data to identify the accuracy level is gradually decreasing over a number of time periods (e.g., from eighty-five percent at a first time period, to eighty-four percent at a second time period, to eighty-three percent at a third time period, and so on). In this manner, the prediction data associated with the prediction performance may be analyzed to determine whether the oxygen saturation model does or does not meet the performance metric and/or is expected not to meet the performance metric over time.

Further, in response to determining that the prediction performance does not meet the performance metric (or is expected not to meet the performance metric over time), the computing system 210 may retrain the oxygen saturation prediction model using ground truth data obtained during the one or more patient monitoring sessions to generate an updated oxygen saturation model (706). Indeed, the computing system 210 may incorporate the ground truth data into a training set to retrain the oxygen saturation prediction model. The ground truth data may include stored or real-time data associated with an actual oxygen saturation level of the patient. In some embodiments, the oxygen saturation model may also be retrained based on a heart rate of the patient, blood pressure of the patient, demographics of the patient, and/or one or more metrics derived from PPG signals of the patient. As such, the computing system 210 may enable an improvement in the accuracy level of the predictions made by the updated oxygen saturation prediction model by retraining the oxygen saturation prediction model.

In some embodiments, the computing system 210 may freeze a layer of the oxygen saturation prediction model, which may prevent weights of that layer from being updated during training. Additionally or alternatively, the computing system 210 may use a learning rate (e.g., a small learning rate) to perform the retraining of the oxygen saturation prediction model based on a portion of the ground truth data that caused the prediction performance of the oxygen saturation prediction model to not meet the performance metric. The learning rate may be associated with a size of a step the oxygen saturation prediction model implements when learning from the data. Therefore, the computing system 210 may enable the oxygen saturation prediction model to update (e.g., adjust, adapt, self-correct) in real-time or at any other suitable time, on any suitable site, without input from a user (e.g., without manually labeling and inputting data). Indeed, the computing system 210 may enable the oxygen saturation prediction model to update (e.g., adjust, adapt, self-correct) in real-time or at any other suitable time while in use on a particular patient (e.g., to be more effective and appropriately reduce nuisance alarms for the particular patient).

The computing system 210 may then track, across an additional plurality of predictions during one or more additional patient monitoring sessions, prediction performance of the updated oxygen saturation model used by the one or more patient monitoring devices for the one or more patients, one or more additional patients, or both (708). In this manner, the computing system 210 may utilize the updated oxygen saturation model to make predictions (e.g., to reduce nuisance alerts) and may also assess the updated oxygen saturation model to detect whether additional adjustments may be implemented to correct additional inaccuracies in the prediction performance of the updated oxygen saturation prediction model. In some embodiments, the updated oxygen saturation prediction model may be compared to a base oxygen saturation prediction model (e.g., running in the background) to enable the computing system 210 to determine whether the updated oxygen saturation model is outperforming the base oxygen saturation model, and to make appropriate adjustments or take corrective actions (e.g., switch to the base oxygen saturation prediction model).

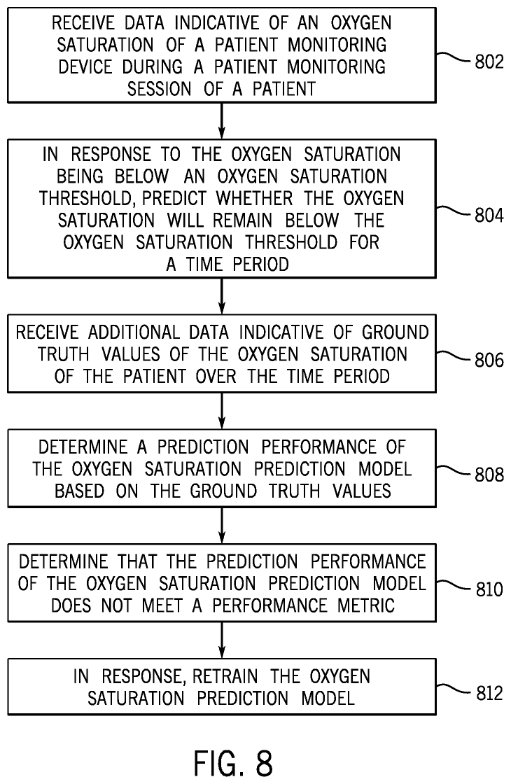

FIG. 8 is a flow diagram illustrating an example method for retraining an oxygen saturation prediction model to increase accuracy of predictions made by patient monitoring devices. Although FIG. 8 is described with respect to computing system 210 (FIG. 2), in other examples, different processing circuitry, alone or in combination with the computing system 210, may perform any part of the technique of FIG. 8.

As shown in FIG. 8, computing system 210 may receive data indicative of an oxygen saturation of a patient monitoring device during a patient monitoring session of a patient (802). For example, the patient monitoring device may include one or more oxygen saturation sensing devices configured to generate a PPG, where the PPG indicates blood oxygen saturation of the patient. The computing system 210 may then, in response to the oxygen saturation being below an oxygen saturation threshold, predict whether the oxygen saturation will remain below the oxygen saturation threshold for a time period (804).

Further, the computing system 210 may receive additional data indicative of ground truth values of the oxygen saturation of the patient over the time period (806). Indeed, the ground truth values may be associated with the actual oxygen saturation level of the patient. The computing system 210 may then determine a prediction performance of the oxygen saturation prediction model based on the ground truth values (808). As an example, the computing system 210 may compare the ground truth values to the prediction (e.g., whether the oxygen saturation will remain below the oxygen saturation threshold) made by the computing system 210 to identify whether the ground truth values are consistent with the prediction.

The computing system 210 may then determine that the prediction performance of the oxygen saturation prediction model does not meet a performance metric (810). As described herein, the performance metric may include an accuracy level, a specificity level, and/or a sensitivity level. Therefore, as an example, determining the prediction performance does not meet the performance metric may include comparing the accuracy level, the specificity level, and/or the sensitivity level to separate thresholds for accuracy, specificity, and/or sensitivity implemented or considered to evaluate the prediction performance. In response to determining that the prediction performance does not meet the performance metric, the computing system 210 may retrain the oxygen saturation prediction model (812). That is, the computing system 210 may incorporate the ground truth values (e.g., the stored or real-time data of the oxygen saturation of the patient) into a training set to retrain the oxygen saturation prediction model. In this manner, the computing system 210 may enable an improvement in the prediction performance of the oxygen saturation prediction model by retraining the oxygen saturation prediction model based on the ground truth values.

The techniques described in this disclosure, including those attributed to computing system 210, oxygen saturation prediction system 220, training system 230, and patient monitoring devices 202, may be implemented, at least in part, in hardware, software, firmware or any combination thereof. For example, various aspects of the techniques may be implemented within one or more processors, including one or more microprocessors, DSPs, ASICS, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, FRAM, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

Various examples of the disclosure have been described. Any combination of the described systems, operations, or functions is contemplated. These and other examples are within the scope of the following claims.

What is claimed is:

1. A method comprising:
tracking, with processing circuitry of a computing system and across a plurality of predictions, prediction performance of a first oxygen saturation prediction model used by one or more patient monitoring devices by comparing a respective prediction made by the first oxygen saturation prediction model to a corresponding ground truth;
determining, with the processing circuitry, that the prediction performance of the first oxygen saturation prediction model does not meet a performance metric, wherein the performance metric comprises an accuracy level, a specificity level, a sensitivity level, or any combination thereof; and
in response to determining that the prediction performance of the first oxygen saturation prediction model does not meet the performance metric, causing, by the processing circuitry, the one or more patient monitoring devices to use a second oxygen saturation prediction model to predict future oxygen saturation levels of one or more patients.

2. The method of claim 1, wherein:
tracking the prediction performance of the first oxygen saturation prediction model comprises tracking, with the processing circuitry and across the plurality of predictions, the accuracy level of the first oxygen saturation prediction model, and
determining the prediction performance of the first oxygen saturation prediction model does not meet the performance metric based on determining, with the processing circuitry, that the accuracy level of the first oxygen saturation prediction model is less than or equal to a minimum accuracy threshold.

3. The method of claim 2, comprising:
storing, with the processing circuitry, for each of the plurality of predictions, an indication of whether the respective prediction made by the first oxygen saturation prediction model was accurate, corresponding input data used by the first oxygen saturation prediction model to make the respective prediction, and the corresponding ground truth of the respective prediction in a memory.

4. The method of claim 1, wherein at least one patient monitoring device of the one or more patient monitoring devices are configured to execute a plurality of oxygen saturation prediction models to make predictions of future oxygen saturation levels of the one or more patients, the method further comprising:
selecting, with the processing circuitry, the second oxygen saturation prediction model from the plurality of oxygen saturation prediction models to replace the first oxygen saturation prediction model.

5. The method of claim 4, wherein selecting the second oxygen saturation prediction model from the plurality of oxygen saturation prediction models comprises:
tracking, with the processing circuitry, a corresponding prediction performance of each oxygen saturation prediction model of the plurality of oxygen saturation prediction models across a corresponding plurality of predictions; and
selecting, with the processing circuitry and based on the corresponding prediction performance of the second oxygen saturation prediction model, the second oxygen saturation prediction model from the plurality of oxygen saturation prediction models to replace the first oxygen saturation prediction model.

6. The method of claim 1, wherein causing the one or more patient monitoring devices to use the second oxygen saturation prediction model to predict the future oxygen saturation levels of the one or more patients comprises:
updating, with the processing circuitry, the first oxygen saturation prediction model using data associated with incorrect predictions made by the first oxygen saturation prediction model to generate an updated oxygen saturation prediction model; and
causing, with the processing circuitry, the one or more patient monitoring devices to use the updated oxygen saturation prediction model to predict the future oxygen saturation levels of the one or more patients.

7. The method of claim 6, wherein updating the first oxygen saturation prediction model comprises:
updating, with the processing circuitry, the first oxygen saturation prediction model based on data specific to a particular patient to generate the updated oxygen saturation prediction model that is specific to the particular patient.

8. The method of claim 6, wherein updating the first oxygen saturation prediction model comprises:
updating, with the processing circuitry, the first oxygen saturation prediction model based on data specific to a particular patient demographic to generate the updated oxygen saturation prediction model that is specific to the particular patient demographic.

9. The method of claim 6, wherein updating the first oxygen saturation prediction model comprises:
updating, with the processing circuitry, the first oxygen saturation prediction model based on data specific to a particular time period to generate the updated oxygen saturation prediction model that is specific to the particular time period.

10. The method of claim 6, wherein updating the first oxygen saturation prediction model comprises:
updating, with the processing circuitry, the first oxygen saturation prediction model based on a particular hospital ward to generate the updated oxygen saturation prediction model that is specific to the particular hospital ward.

11. The method of claim 1, wherein the first oxygen saturation prediction model is trained to be a patient-specific oxygen saturation prediction model, and wherein an electronic medical record of a patient includes data for generating the patient-specific oxygen saturation prediction model.

12. A computing system comprising:
processing circuitry; and
memory storing instructions that, when executed by the processing circuitry, cause the processing circuitry to:
track, across a plurality of predictions, prediction performance of a first oxygen saturation prediction model used by one or more patient monitoring devices by comparing a respective prediction made by the first oxygen saturation prediction model to a corresponding ground truth;
determine that the prediction performance of the first oxygen saturation prediction model does not meet a performance metric, wherein the performance metric comprises an accuracy level, a specificity level, a sensitivity level, or any combination thereof; and
in response to determining that the prediction performance of the first oxygen saturation prediction model does not meet the performance metric, cause the one or more patient monitoring devices to use a second oxygen saturation prediction model to predict future oxygen saturation levels of one or more patients.

13. The computing system of claim 12, wherein the instructions, when executed by the processing circuitry, cause the processing circuitry to:
track, across the plurality of predictions, the accuracy level of the first oxygen saturation prediction model to track the prediction performance of the first oxygen saturation prediction model; and
determine that the accuracy level of the first oxygen saturation prediction model is less than or equal to a minimum accuracy threshold to determine that the prediction performance of the first oxygen saturation prediction model does not meet the performance metric.

14. The computing system of claim 12, wherein the instructions, when executed by the processing circuitry, cause the processing circuitry to:
determine, for each of the plurality of predictions made by the first oxygen saturation prediction model, whether the respective prediction made by the first oxygen saturation prediction model was accurate, corresponding input data used by the first oxygen saturation prediction model to make the respective prediction, and the ground truth of the respective prediction to track the prediction performance of the first oxygen saturation prediction model.

15. The computing system of claim 14, wherein the instructions, when executed by the processing circuitry, cause the processing circuitry to:
store, for each of the plurality of predictions, an indication of whether the respective prediction made by the first oxygen saturation prediction model was accurate, the corresponding input data used by the first oxygen saturation prediction model to make the respective prediction, and the ground truth of the respective prediction.

16. The computing system of claim 12, wherein a plurality of oxygen saturation prediction models execute to make background predictions of the future oxygen saturation levels of the one or more patients, and wherein the instructions, when executed by the processing circuitry, cause the processing circuitry to:
select the second oxygen saturation prediction model from the plurality of oxygen saturation prediction models to replace the first oxygen saturation prediction model.

17. The computing system of claim 16, wherein the instructions, when executed by the processing circuitry, cause the processing circuitry to:
track a corresponding prediction performance of each of the plurality of oxygen saturation prediction models across a corresponding plurality of predictions; and
select, based on the corresponding prediction performance of the second oxygen saturation prediction model, the second oxygen saturation prediction model from the plurality of oxygen saturation prediction models to replace the first oxygen saturation prediction model.

18. The computing system of claim 12, wherein the instructions, when executed by the processing circuitry, cause the processing circuitry to:
update the first oxygen saturation prediction model using data associated with incorrect predictions made by the first oxygen saturation prediction model to generate an updated oxygen saturation prediction model; and
cause the one or more patient monitoring devices to use the updated oxygen saturation prediction model as the second oxygen saturation prediction model to predict the future oxygen saturation levels of the one or more patients.

19. The computing system of claim 18, wherein the instructions, when executed by the processing circuitry, cause the processing circuitry to:
update the first oxygen saturation prediction model to generate the updated oxygen saturation prediction model based on data specific to a particular patient.

20. The computing system of claim 18, wherein the instructions, when executed by the processing circuitry, cause the processing circuitry:
update the first oxygen saturation prediction model to generate the updated oxygen saturation prediction model based on data specific to a particular patient demographic, a particular time period, a particular hospital ward, or any combination thereof.
